(19) United States
(12) Patent Application Publication
(10) Pub. No.: US 2025/0136535 A1
(43) Pub. Date: May 1, 2025

(54) HETEROGENEOUS BIMETALLIC CATALYST, METHOD FOR PREPARING SAME AND USE THEREOF IN THE SYNTHESIS OF ETHYLENE GLYCOL FROM CARBON MONOXIDE

(71) Applicant: FAIRBRICS, CLICHY (FR)

(72) Inventors: Ibrahim ABDELLAH, Montigny Les Metz (FR); Yannick BIDAL, POIGNY LA FORÊT (FR); Tawfiq NASR ALLAH, Paris (FR)

(73) Assignee: FAIRBRICS, CLICHY (FR)

(21) Appl. No.: 18/719,496

(22) PCT Filed: Dec. 13, 2022
(86) PCT No.: PCT/EP2022/085704

§ 371 Date: Jun. 13, 2024

(30) Foreign Application Priority Data
Dec. 13, 2021 (FR) 2113380

(51) Int. Cl. C07C 29/157; B01J 23/89; B01J 35/61

(52) U.S. Cl. CPC C07C 29/157; B01J 23/8926; B01J 35/61; B01J 2523/17; B01J 2523/18; B01J 2523/48; B01J 2523/824; C07C 2523/89

(57) ABSTRACT

A supported bimetallic catalyst according to the formula Pd-M/Support, which includes palladium and a metal M on a support, wherein M represents Cu or Ag, for use in a method for preparing ethylene glycol from an alcohol. The method includes two reaction steps catalyzed by the bimetallic catalyst of formula Pd-M/Support.

The present invention concerns a novel heterogeneous bimetallic catalyst, method for preparing the same and use thereof in the synthesis of ethylene glycol from carbon monoxide.

### CONTEXT OF THE INVENTION

Ethylene glycol is an important material in the chemical industry, enabling the production of textile fibres and polyester resins. One way of synthesizing ethylene glycol is to hydrogenate oxalates.

Oxalates are high value-added raw materials in the chemical industry. They are used on a large scale to produce various dyes, medicines, solvents, extraction agents and various intermediates in the fine chemicals industry.

A traditional production method for oxalates involves the esterification of oxalic acid with alcohols. This production technique is costly, energy-intensive, polluting and involves unreasonable use of raw materials.

In general, the carbonylation reaction from CO leading to the formation of oxalate, and the hydrogenation reaction from oxalate to ethylene glycol are catalysed by two quite different catalysts. For example, the carbonylation step is generally carried out with a heterogeneous palladium-based catalyst, while the hydrogenation step is carried out with a copper-based catalyst. However, copper does not catalyse the formation of oxalate, and palladium does not allow the hydrogenation of oxalic derivatives to ethylene glycol. The two steps therefore use very different catalysts with different transition metals, which can present a major challenge in a large-scale industrial process.

For many years, there has been a need for a low-cost, environmentally-friendly way of producing ethylene glycol.

Until now, it is looking for a heterogeneous catalyst that will enable the efficient synthesis of ethylene glycol in a way that is environmentally friendly, easy to industrialize and safe.

The use of a catalyst capable of both catalysing the reaction of carbonylation of alcohol to oxalate and also catalysing the reaction of hydrogenation of oxalate to ethylene glycol in a process for preparing ethylene glycol is of industrial interest and advantage in practical and material terms.

One of the aims of the invention is to propose a process for preparing ethylene glycol, in two catalysed reaction steps using a single catalyst of the same nature, the two reaction steps being the carbonylation of alcohol to oxalate and the hydrogenation of oxalate to ethylene glycol.

Another aim of the invention is a process for preparing ethylene glycol in two reaction steps of carbonylation and hydrogenation with efficient yields and high selectivity.

Another aim of the invention is to prepare ethylene glycol without using toxic reagents such as nitro-derivatives.

Another aim of the invention is the preparation of ethylene glycol, using recyclable or recycled reagents such as CO₂ or CO and an efficient and reusable heterogeneous catalyst.

Another aim of the invention is to provide a new heterogeneous supported bimetallic catalyst.

Another aim of the invention is to provide a heterogeneous catalyst that can be used in a continuous flow process.

Another aim of the present invention is to propose a simple, suitable for industry and optimised preparation process for this catalyst.

A first object of the present invention is the use of a supported bimetallic catalyst of formula Pd-M/Support, comprising palladium and a metal M on a support, in which M represents Cu or Ag, in the implementation of a process for preparing ethylene glycol from an alcohol, comprising two reaction steps catalysed by the same bimetallic catalyst, in particular
wherein the first catalysed reaction step is an oxidative carbonylation, from an alcohol, carbon monoxide and an oxidant, in particular molecular oxygen, optionally in the presence of a promoter, to form an oxalate compound as reaction intermediate, and
in which the second catalysed reaction step is a hydrogenation reaction of said oxalate compound by hydrogen to obtain ethylene glycol.

For the purposes of this invention, “same catalyst” means a catalyst of the same nature, the characteristics of which are identical to those of the catalyst referred to, i.e. in particular the physical, chemical and surface characteristics, in particular the nature of the metals and the support, their proportions, the size and morphology of the particles.

The inventors have surprisingly observed the possibility of using supported bimetallic palladium-copper or palladium-silver catalysts to catalyse the reaction of carbonylation of alcohol to oxalate and the reaction of hydrogenation of oxalate to ethylene glycol. Both reactions can be carried out without prior treatment of the catalyst by a step of metal reduction under hydrogen. The combination of palladium with copper or silver (two metals in the same column as gold in the Periodic Table of the Elements) makes it possible to obtain catalysts capable of catalysing the carbonylation and hydrogenation reactions implemented in the process for preparing ethylene glycol from an alcohol.

According to a particular embodiment, the invention relates to the use of a supported bimetallic catalyst of formula Pd—Cu/Support, in the implementation of a method for preparing ethylene glycol from an alcohol, comprising two reaction steps catalysed by the same bimetallic catalyst, in particular:
wherein the first catalysed reaction step is an oxidative carbonylation, from an alcohol, carbon monoxide and an oxidant, in particular molecular oxygen, optionally in the presence of a promoter, to form an oxalate compound as reaction intermediate, and
wherein the second catalysed reaction step is a hydrogenation reaction of the said oxalate compound by hydrogen to obtain ethylene glycol.

The inventors also unexpectedly observed that the use of the bimetallic Pd—Cu/ZrO₂ catalyst made it possible to obtain higher yields of the oxidative carbonylation and hydrogenation reaction steps in the preparation of ethylene glycol than those obtained with the corresponding monometallic Pd/ZrO₂ and Cu/ZrO₂ catalysts, demonstrating a synergistic effect of the bimetallic catalyst for carbonylation and hydrogenation.

According to a particular embodiment, the invention relates to the use of a supported bimetallic catalyst of formula Pd—Ag/Support, in the implementation of a process for the preparation of ethylene glycol from an alcohol, comprising two reaction steps catalysed by the same bimetallic catalyst, in particular:
wherein the first catalysed reaction step is an oxidative carbonylation, from an alcohol, carbon monoxide and an oxidant, in particular molecular oxygen, optionally in the presence of a promoter, to form an oxalate compound as reaction intermediate, and
in which the second catalysed reaction step is a hydrogenation reaction of the said oxalate compound by hydrogen to obtain ethylene glycol.

The invention is based on the use of the same catalyst, i.e. a catalyst with the same characteristics, capable of catalysing two different reaction steps used in the ethylene glycol preparation process, in particular an oxidative carbonylation reaction step and a hydrogenation step.

For the purposes of this invention, “Pd-M bimetallic catalyst” means a catalyst in which the catalytic sites comprise palladium and a metal M, M being Cu or Ag. The palladium and the metal M combine to form the catalytic sites operating during the two catalysed reaction steps. Bimetallic catalysts according to the invention include catalysts in which the two metals are in the form of a core-shell structure, or in the form of a homogeneous alloy, or in the form of two distinct populations of particles of palladium and of metal M respectively, or in the form of a mixture of populations of these different forms.

It is understood that the Pd-M/Support catalyst is a heterogeneous catalyst.

The use of a heterogeneous catalyst has the advantage of facilitating the separation of the catalyst from the other species involved in the reaction, making it easy to recover and reuse the catalyst.

The use of a heterogeneous catalyst also has the advantage of making it possible, in the reactor, to fix the catalyst in an enclosure such as a cartridge when operating under continuous flow, and thus to obtain catalyst-free products at the reactor outlet.

For the purposes of this invention, “reaction step” means a synthesis step involving a chemical reaction using starting reagents to form end products. The chemical reaction leads to a transformation of the molecular structure of the starting reagents. Carbonylation and hydrogenation are understood to be reaction steps. On the other hand, product recovery, washing or purification steps are not considered reaction steps.

A reaction step is said to be catalysed when it requires a catalyst for its implementation.

For the purposes of this invention, “same catalyst” means a catalyst of the same nature, with the same characteristics such as the nature of the metals and the support, and their proportions.

Thus, one of the industrial advantages is that only one type of catalyst can be used or prepared, limiting the raw materials or catalyst preparation steps required for the ethylene glycol preparation process. Limiting the number of raw materials means that purchasing costs and waste management can be optimised. Limiting the number of industrial steps is advantageous from the point of view of costs and process implementation.

In a particular embodiment, the invention relates to the use as defined above, in which the catalyst support is an oxide.

Oxide supports are commonly used to prepare heterogeneous catalysts, partly because of the efficiency of the catalytic properties obtained and partly because they are readily available and easy to prepare. For example, they are advantageously less expensive than polymer supports.

In a particular embodiment, the invention relates to the use as defined above, in which the catalyst support is an oxide selected from zirconium dioxide ZrO₂, alumina Al₂O₃, silica SiO₂, cerium dioxide CeO₂, titanium dioxide TiO₂, magnesium oxide MgO, indium oxide In₂O₃, or a mixture of these oxides, preferably zirconium dioxide ZrO₂.

In a particular embodiment, the invention relates to the use as defined above, in which the catalyst support is an oxide selected from zirconium dioxide ZrO₂, alumina Al₂O₃ and silica SiO₂.

In a particular embodiment, the invention relates to the use as defined above, wherein said supported bimetallic catalyst is a Pd—Cu/ZrO₂ catalyst.

In a particular embodiment, the zirconium dioxide support ZrO₂ has a melting temperature of 2700 to 2750° C., in particular 2715° C., and a density of 5 to 6 g/cm³.

The zirconium dioxide support can, for example, be obtained from STERM Chemicals (15 Rue de l'Atome, 67800 Bischheim).

The Pd—Cu bimetallic catalyst is preferably supported on zirconium dioxide ZrO₂, but another type of support based on another oxide such as Al₂O₃, SiO₂, CeO₂, TiO₂, MgO, In₂O₃ can also be implemented.

In a particular embodiment, the oxide support of said Pd-M bimetallic catalyst is in the form of a layer on the surface of another inert material, i.e. non-chemically active, such as ceramics or glasses.

In a particular embodiment, the invention relates to the use as defined above, in which the catalyst has a palladium content from 0.1 to 10%, in particular 2%, and a content of metal M from 0.1 to 40%, in particular 10% or 15%, by weight relative to the total weight of the catalyst. It is understood that the ratio between the total content of metals Pd-M and the support varies from 0.2 to 50% by weight in the catalysts of the invention. The weight of the metals represents up to half the total weight of the catalyst.

The expression “from 0.1 to 10%” corresponds to the following ranges: from 0.1 to 1%; from 1 to 2%; from 2 to 3%; from 3 to 4%; from 4 to 5%; from 5 to 6%; from 6 to 7%; from 7 to 8%; from 8 to 9%; from 9 to 10%.

The expression “from 0.1 to 40%” corresponds to the following ranges: from 0.1 to 5%; from 5 to 10%; from 10 to 15%; from 15 to 20%; from 20 to 25%; from 25 to 30%; from 30 to 35%; from 35 to 40%.

In a particular embodiment, the invention relates to the use as defined above, in which the weight ratio between Pd and M is from 1:1 to 1:20, preferably from 1:1 to 1:10, more preferably 1:5.

The expression “from 1:1 to 1:20” corresponds to the ranges: from 1:1 to 1:2; from 1:2 to 1:3; from 1:3 to 1:4; from 1:4 to 1:5; from 1:5 to 1:6; from 1:6 to 1:7; from 1:7 to 1:8; from 1:8 to 1:9; from 1:9 to 1:10; 1:10 to 1:11; from 1:11 to 1:12; from 1:12 to 1:13; from 1:13 to 1:14; from 1:14 to 1:15; from 1:15 to 1:16; from 1:16 to 1:17; from 1:17 to 1:18; from 1:18 to 1:19; from 1:19 to 1:20.

In a particular embodiment, the invention relates to the use as defined above, wherein the first catalysed reaction step is an oxidative carbonylation, from an alcohol, carbon monoxide and an oxidant, in particular molecular oxygen, optionally in the presence of a promoter, to form an oxalate compound as reaction intermediate, and wherein the second catalysed reaction step is a hydrogenation reaction of said oxalate compound by hydrogen to obtain ethylene glycol. The general diagram of a preparation process according to the invention can be represented as follows:

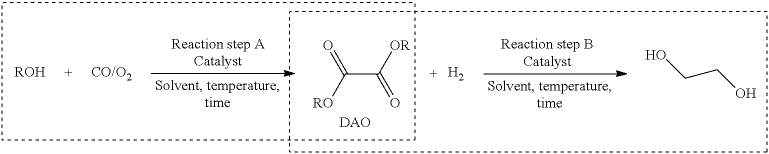

By “oxalate” it is meant the dialkyloxalate (DAO) corresponding to the alcohol used.

By “promoter” or “reaction promoter” it is meant a substance that can improve the properties of a catalyst, such as catalytic activity, selectivity, stability, lifetime or prevent deactivation of the catalyst.

In a particular embodiment, the promoter is an oxidant. The promoter can thus promote the oxidative carbonylation process.

According to a particular embodiment, the invention relates to the use as defined above, the implementation of the oxidative carbonylation reaction step comprising at least one additive.

By “additive” it is meant a substance which improves the yield of the reaction but which is not essential for it to take place. An additive does not intervene directly in the transformation of the substrate and consequently does not enter into the balance equation of the reaction.

According to a particular embodiment, the additive is a base, preferably selected from triethylamine (Et₃N), 2,6-lutidine, caesium carbonate (Cs₂CO₃), or 1-methylimidazole.

Another object of the present invention relates to a method of preparation of ethylene glycol comprising:
a first reaction step A of oxidative carbonylation of an alcohol, in the presence of a supported bimetallic catalyst of formula Pd-M/Support in which M represents Cu or Ag, to obtain an oxalate compound as reaction intermediate, and
a second reaction step B of hydrogenation of said oxalate compound, optionally purified, produced in reaction step A, to ethylene glycol, in the presence of said catalyst of formula Pd-M/Support.

The reaction step A, corresponding to the oxidative carbonylation reaction of an alcohol, is schematised as follows:

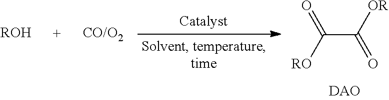

The reaction step B corresponding to the hydrogenation of an oxalate compound (DAO) is schematised as follows:

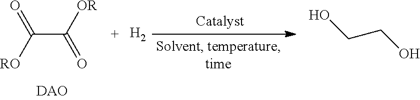

According to a particular embodiment, the invention relates to a method of preparation of ethylene glycol as defined above, in which:
reaction step A comprises
   bringing into contact:
      an alcohol, in particular selected from methanol and ethanol,
      carbon monoxide,
      an oxidant, in particular oxygen O₂,
      a catalyst of formula Pd-M/Support in which M represents Cu or Ag, in particular a catalyst comprising an oxide support selected from zirconium dioxide ZrO₂, alumina Al₂O₃, silica SiO₂, cerium dioxide CeO₂, titanium dioxide TiO₂, magnesium oxide MgO, indium oxide In₂O₃, or a mixture of these oxides, preferably a catalyst of formula Pd—Cu/ZrO₂;
      optionally a promoter, in particular an iodine compound, notably selected from tetramethylammonium iodide, potassium iodide or sodium iodide, preferably tetramethylammonium iodide,
      optionally a base, in particular triethylamine,
      optionally a solvent, in particular selected from acetonitrile, tetrahydrofuran, dioxane and toluene, preferably acetonitrile;
   to obtain a reaction medium 1 which may be pressurised from 0.1 to 15 MPa,
   optionally heating said reaction medium 1 to a temperature from 25 to 200° C., preferably about 90° C., in particular during 2 to 24 hours, preferably 16 hours,
to obtain the oxalate compound;
reaction step B comprises
   bringing into contact:
      said oxalate compound,
      dihydrogen,
      said catalyst of formula Pd-M/Support in which M represents Cu or Ag,
      optionally a solvent, in particular ethanol, methanol and dioxane, preferably ethanol or methanol.
to obtain a reaction medium 2 which may be pressurised from 0.1 to 15 MPa, in particular 5 MPa,
   optionally heating said reaction medium 2 to a temperature from 100 to 250° C., in particular 200 or 220° C., preferably during 5 to 24 hours, more preferably during 8 or 16 hours,
to obtain ethylene glycol.

According to a particular embodiment, the invention relates to a method as defined above, in which:
reaction step A comprises
   bringing into contact:
      an alcohol,
      carbon monoxide,
      an oxidant,
      a catalyst of formula Pd-M/Support in which M represents Cu or Ag,
      optionally a promoter,
      optionally a base,
      optionally a solvent,
   to obtain a reaction medium 1 which may be pressurised from 0.1 to 15 MPa,
   optionally heating the reaction medium,
to obtain the oxalate compound;
reaction step B comprises
   bringing into contact:
      said oxalate compound
      dihydrogen,
      said catalyst of formula Pd-M/Support in which M represents Cu or Ag,
      optionally a solvent,
   to obtain a reaction medium 2 which may be pressurised from 0.1 to 15 MPa, in particular 5 MPa,
   optionally heating said reaction medium 2,
to obtain ethylene glycol.

### Solvent

The method according to the present invention has the advantage of not requiring a solvent in reaction step A, the oxidative carbonylation of the alcohol. Advantageously, the alcohol can act both as reagent and solvent in reaction step A.

According to a particular embodiment, the invention relates to a method as defined above, in which:
reaction step A comprises
   bringing into contact:
      an alcohol,
      carbon monoxide,
      an oxidant,
      a catalyst of formula Pd-M/Support in which M represents Cu or Ag,
      a promoter,
      a base,
      optionally a solvent,
   to obtain a reaction medium 1 pressurised from 0.1 to 15 MPa, in particular 8 MPa;
   heating said reaction medium,
to obtain the oxalate compound;
reaction step B comprises
   bringing into contact:
      said oxalate compound
      dihydrogen,
      said catalyst of formula Pd-M/Support in which M represents Cu or Ag,
      a solvent,
   to obtain a reaction medium 2 pressurised from 0.1 to 15 MPa, in particular 5 MPa,
   heating said reaction medium 2,
to obtain ethylene glycol.

Advantageously, oxidative carbonylation reaction step A of the process according to the invention can be carried out in the absence of solvent.

According to a particular embodiment, the invention relates to a method as defined above, in which:
reaction step A comprises
   bringing into contact:
      an alcohol,
      carbon monoxide,
      an oxidant,
      a catalyst of formula Pd-M/Support in which M represents Cu or Ag,
      a promoter,
      a base,
   to obtain a reaction medium 1 pressurised from 0.1 to 15 MPa, in particular 8 MPa,
   heating said reaction medium,
to obtain the oxalate compound;
reaction step B comprises
   bringing into contact:
      said oxalate compound
      dihydrogen,
      said catalyst of formula Pd-M/Support in which M represents Cu or Ag,
      a solvent,
to obtain a reaction medium 2 which may be pressurised from 0.1 to 15 MPa, in particular 5 MPa,
   heating said reaction medium,
to obtain ethylene glycol.

Advantageously, oxidative carbonylation reaction step A of the method according to the invention can be carried out in the presence of a solvent.

According to a particular embodiment, the invention relates to a method as defined above, in which:
reaction step A comprises
   bringing into contact:
      an alcohol,
      carbon monoxide,
      a base,
      an oxidant,
      a catalyst of formula Pd-M/Support in which M represents Cu or Ag,
      a promoter,
      a solvent,
   to obtain a reaction medium 1 pressurised from 0.1 to 15 MPa, in particular 8 MPa,
   heating said reaction medium,
to obtain the oxalate compound;
reaction step B comprises
   bringing into contact:
      said oxalate compound
      dihydrogen,
      said catalyst of formula Pd-M/Support in which M represents Cu or Ag,
      a solvent,
   to obtain a reaction medium 2 pressurised from 0.1 to 15 MPa, in particular 5 MPa,
   heating said reaction medium,
to obtain ethylene glycol.

By “reaction medium” it is meant all the species brought together during a chemical reaction. It includes in particular the reagents in liquid or gaseous form, the catalyst, and optionally a solvent, additives or promoters.

The expression MPa corresponds to 106 Pascal and is equivalent to 10 bars.

The expression “from 0.1 to 15.0 MPa” corresponds to the ranges: from 0.1 to 0.5 MPa; from 0.5 to 1.0 MPa; from 1.0 to 1.5 MPa; from 1.5 to 2.0 MPa; from 2.0 to 2.5 MPa; from 2.5 to 3.0 MPa; from 3.0 to 3.5 MPa; from 3.5 to 4.0 MPa; from 4.0 to 4.5 MPa; from 4.5 to 5.0 MPa; from 5.0 to 5.5 MPa; from 5.5 to 6.0 MPa; from 6.0 to 6.5 MPa; from 6.5 to 7.0 MPa; from 7.0 to 7.5 MPa; from 7.5 to 8.0 MPa; from 8.0 to 8.5 MPa; from 8.5 to 9.0 MPa; from 9.0 to 9.5 MPa; from 9.5 to 10.0 MPa; from 10.0 to 10.5 MPa; from 10.5 to 11.0 MPa; from 11.0 to 11.5 MPa; from 11.5 to 12.0 MPa; from 12.0 to 12.5 MPa; from 12.5 to 13.0 MPa; from 13.0 to 13.5 MPa; from 13.5 to 14.0 MPa; from 14.0 to 14.5 MPa; from 14.5 to 15.0 MPa.

According to a particular embodiment, the invention relates to a method as defined above, in which the alcohol used in reaction step A is also used as solvent in reaction step B. Advantageously, it is thus not necessary to evaporate the alcohol in the oxalate product obtained in reaction step A before using it as a reagent in reaction step B.

### Base

The method according to the present invention has the advantage of not requiring a base in reaction step A, oxidative carbonylation of the alcohol. Indeed advantageously the base is an additive which promotes the efficiency of the reaction but which is not necessary for carrying out reaction step A.

According to a particular embodiment, the invention relates to a method as defined above, in which:
reaction step A comprises
   bringing into contact:
      an alcohol,
      carbon monoxide,
      an oxidant,
      a catalyst of formula Pd-M/Support in which M represents Cu or Ag,
      a promoter,
      optionally a base,
      a solvent,
   to obtain a reaction medium 1 pressurised from 0.1 to 15 MPa, in particular 8 MPa;
   heating said reaction medium,
to obtain the oxalate compound;
reaction step B comprises
   bringing into contact:
      said oxalate compound
      dihydrogen,
      said catalyst of formula Pd-M/Support in which M represents Cu or Ag,
      of a solvent,
   to obtain a reaction medium 2 pressurised from 0.1 to 15 MPa, in particular 5 MPa,
   heating said reaction medium 2,
to obtain ethylene glycol.

Advantageously, oxidative carbonylation reaction step A of the method according to the invention can be carried out without a base.

According to a particular embodiment, the invention relates to a method as defined above, in which:
reaction step A comprises
   bringing into contact:
      an alcohol,
      carbon monoxide,
      an oxidant,
      a catalyst of formula Pd-M/Support in which M represents Cu or Ag,
      a promoter,
      of a solvent,
   to obtain a reaction medium 1 pressurised from 0.1 to 15 MPa, in particular 8 MPa;
   heating said reaction medium,
to obtain the oxalate compound;
reaction step B comprises
   bringing into contact:
      said oxalate compound
      dihydrogen,
      said catalyst of formula Pd-M/Support in which M represents Cu or Ag,
      a solvent,
   to obtain a reaction medium 2 pressurised from 0.1 to 15 MPa, in particular 5 MPa,
   heating said reaction medium 2,
to obtain ethylene glycol.

### Other Process Parameters

According to a particular embodiment, the invention relates to a method as defined above, in which said catalyst comprises an oxide support selected from zirconium dioxide ZrO₂, alumina Al₂O₃, silica SiO₂, cerium dioxide CeO₂, titanium dioxide TiO₂, magnesium oxide MgO, indium oxide In₂O₃, or a mixture of these oxides, preferably zirconium dioxide ZrO₂.

According to a particular embodiment, the invention relates to a method as defined above, in which said catalyst is of formula Pd—Cu/ZrO₂.

According to a particular embodiment, the invention relates to a method as defined above, in which the catalyst has a palladium content from 0.1 to 10%, in particular 2%, and a content of metal M from 0.1 to 40%, in particular 10%, by weight relative to the total weight of the catalyst.

According to a particular embodiment, the invention relates to a method as defined above, in which the catalyst is used in reaction step A in a proportion from 0.01 to 10 mol % of palladium relative to the alcohol.

The expression “from 0.01 to 10%” corresponds to the following ranges: from 0.01 to 0.05%; from 0.05 to 0.1%; from 0.1 to 0.15%; from 0.15 to 0.2%; from 0.2 to 0.5%; from 0.5 to 1%; from 1 to 2%; from 2 to 3%; from 3 to 4%; from 4 to 5%; from 5 to 6%; from 6 to 7%; from 7 to 8%; from 8 to 9%; from 9 to 10%.

According to a particular embodiment, the invention relates to a method as defined above, in which carbon monoxide is used in reaction step A at a rate from 0.5 to 8.0 MPa, in particular 6.5 MPa.

The expression “from 0.5 to 8.0 MPa” corresponds to the ranges: from 0.5 to 1.0 MPa; from 1.0 to 1.5 MPa; from 1.5 to 2.0 MPa; from 2.0 to 2.5 MPa; from 2.5 to 3.0 MPa; from 3.0 to 3.5 MPa; from 3.5 to 4.0 MPa; from 4.0 to 4.5 MPa; from 4.5 to 5.0 MPa; from 5.0 to 5.5 MPa; from 5.5 to 6.0 MPa; from 6.0 to 6.5 MPa; from 6.5 to 7.0 MPa; from 7.0 to 7.5 MPa; from 7.5 to 8.0 MPa.

According to a particular embodiment, the invention relates to a method as defined above, in which the oxidant in reaction step A is oxygen, used at a rate from 0.5 to 2.5 MPa, in particular 1.5 MPa.

The expression “from 0.5 to 2.5 MPa” corresponds to the following ranges: from 0.5 to 1.0 MPa; from 1.0 to 1.5 MPa; from 1.5 to 2.0 MPa; from 2.0 to 2.5 MPa.

According to a particular embodiment, the invention concerns a method as defined above in which the oxidant is selected from: molecular oxygen (O₂), air, a dione in particular 1,4-benzoquinone, 1,4-dichloro-2-butene and CuCl₂.

It is understood that air containing 20% O₂ can be used to carry out the process in which O₂ is the oxidant.

According to a particular embodiment, the invention relates to a method as defined above, in which a promoter is used in reaction step A, in particular an iodine compound, in particular selected from tetramethylammonium iodide, potassium iodide or sodium iodide,

According to a particular embodiment, the invention relates to a method as defined above, in which the promoter is used in a proportion from 0.1 to 5 mol % relative to the alcohol, in particular in a proportion of 0.2 mol %.

The expression “from 0.1 to 5%” corresponds to the following ranges: from 0.1 to 0.15%; from 0.15 to 0.2%; from 0.2 to 0.3%; from 0.3 to 0.4%; from 0.4 to 0.5%; from 0.5 to 1%; from 1 to 2%; from 2 to 3%; from 3 to 4%; from 4 to 5%.

According to a particular embodiment, the invention relates to a method as defined above, in which a base is used in reaction step A, in particular triethylamine.

According to a particular embodiment, the invention relates to a method as defined above, in which the base is used in a proportion from 0.1 to 5 mol % relative to the alcohol, in particular in a proportion of 0.15 mol %.

The expression “from 0.1 to 5%” corresponds to the following ranges: from 0.1 to 0.15%; from 0.15 to 0.2%; from 0.2 to 0.3%; from 0.3 to 0.4%; from 0.4 to 0.5%; from 0.5 to 1%; from 1 to 2%; from 2 to 3%; from 3 to 4%; from 4 to 5%.

According to a particular embodiment, the invention relates to a method as defined above, in which a solvent is used in reaction step A, in particular selected from acetonitrile, tetrahydrofuran, dioxane and toluene, preferably acetonitrile.

According to a particular embodiment, the invention relates to a method as defined above, in which in reaction step A, the reaction medium 1 is pressurised from 0.1 to 15 MPa, preferably about 8 MPa.

According to a particular embodiment, the invention relates to a method as defined above, in which in reaction step A, the reaction medium 1 is heated to a temperature from 25 to 200° C., in particular from 60 to 110° C., preferably about 90° C., in particular during from 2 to 24 hours, preferably during 16 hours.

The expression “from 25 to 200° C.” corresponds to the following ranges: from 25 to 40° C.; from 40 to 60° C.; from 60 to 80° C.; from 80 to 100° C.; from 100 to 120° C.; from 120 to 140° C.; from 140 to 160° C.; from 160 to 180° C.; from 180 to 200° C.

The expression “from 60 to 110° C.” corresponds to the following ranges: from 60 to 70° C.; from 70 to 80° C.; from 80 to 90° C.; from 90 to 100° C.; from 100 to 110° C.

The expression “from 2 to 24 hours” corresponds to the following ranges: from 2 to 5 hours; from 5 to 8 hours; from 8 to 12 hours; from 12 to 16 hours; from 16 to 20 hours; from 20 to 24 hours.

In a particular embodiment, the invention relates to a method as defined above, wherein reaction step A comprises bringing into contact an alcohol of Formula 1 to prepare an oxalate compound of Formula 2:

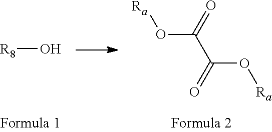

in which Rₐ represents:
a C₁ to C₂₀ linear or branched alkyl group,
a C₃ to C₁₀ cycloalkyl group,
a C₅ to C₂₀ alkyl-aryl or alkyl-heteroaryl group.

For the purposes of this invention, by “C₁ to C₂₀ linear or branched alkyl group” it is meant a saturated, linear or branched, acyclic carbon chain comprising 1 to 20 carbon atoms. These groups are methyl, ethyl, propyl, butyl, pentyl, hexyl, heptyl, octyl, nonyl, decyl, undecyl, dodecyl, tridecyl, tetradecyl, pentadecyl, cetyl, heptadecyl, octadecyl, nonadecyl and eicosyl. The definition of alkyl includes all possible isomers. For example, the term butyl includes n-butyl, iso-butyl, sec-butyl and ter-butyl. One or more hydrogen atoms may be replaced in the alkyl chain.

By “C₃ to C₁₀ cycloalkyl” it is meant: a C₃ cyclopropyl group, a C₄ cyclobutyl group, a C₅ cyclopentyl group, a C₆ cyclohexyl group, a C₇ cycloheptyl group, a C₈ cyclooctyl group, a C₉ cyclononyl group, or a C₁₀ cyclodecyl group, and fused cycloalkane rings such as adamantyl.

By “C₅ to C₂₀ alkylaryl” it is meant a group consisting of a linear or branched alkyl chain linked to an aromatic group, the alkylaryl group comprising 5 to 20 carbon atoms. The aryl groups according to the present invention can also be substituted, in particular by one or more substituents chosen from a C₁ to C₁₀ linear or branched alkyl group.

Phenyl, toluyl, anisyl and naphthyl o-tolyl, m-tolyl, p-tolyl, o-xylyl, m-xylyl, p-xylyl, are examples of aryl groups.

The term “heteroaryl” refers to an aryl group as defined above, comprising atoms other than carbon atoms, in particular N, O or S within the aromatic ring. Pyridyl, imidazoyl, furfuryl or furanyl are examples of heteroaryl groups according to the present invention.

In a particular embodiment, the invention relates to a method as defined above, wherein reaction step A comprises bringing into contact an alcohol of Formula 1 to prepare an oxalate compound of Formula 2:

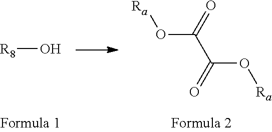

in which Rₐ represents:
a C₁ to C₂₀ linear or branched alkyl group,
a C₃ to C₁₀ cycloalkyl group,
a C₅ to C₂₀ alkyl-aryl or alkyl-heteroaryl group,
in particular selected from methanol, ethanol and isopropanol.

According to a particular embodiment, the invention relates to a method as defined above, in which a purification step of said oxalate compound is carried out between reaction step A and reaction step B, in particular by distillation.

Advantageously, the oxalate compound purification step is carried out by distillation, extraction or recrystallisation in the purification step.

According to a particular embodiment, the invention relates to a method as defined above, in which, in reaction step B, the catalyst is used in an amount from 0.5 to 10 mmol, in particular in an amount of 4 mmol of Cu or Ag.

The expression “from 0.5 to 10 mmol” corresponds to the following ranges: from 0.5 to 1 mmol; from 1 to 2 mmol; from 2 to 3 mmol; from 3 to 4 mmol; from 4 to 5 mmol; from 5 to 6 mmol; from 6 to 7 mmol; from 7 to 8 mmol; from 8 to 9 mmol; from 9 to 10 mmol.

According to a particular embodiment, the invention relates to a method as defined above, in which, in reaction step B, the oxalate compound is used in an amount from 2 to 40 molar equivalents, in particular in a proportion of 5 equivalents, relative to the metal M of the catalyst. The expression “2 to 40 equivalents” corresponds to the following ranges: 2 to 5 equivalents; 5 to 10 equivalents; 10 to 15 equivalents; 15 to 20 equivalents; 20 to 25 equivalents; 25 to 30 equivalents; 30 to 35 equivalents; 35 to 40 equivalents.

According to a particular embodiment, the invention relates to a method as defined above, in which, in reaction step B, dihydrogen is used under a pressure from 1.0 to 8.0 MPa, in particular 5.0 MPa.

The expression “from 1.0 to 8.0 MPa” corresponds to the ranges: from 1.0 to 1.5 MPa; from 1.5 to 2.0 MPa; from 2.0 to 2.5 MPa; from 2.5 to 3.0 MPa; from 3.0 to 3.5 MPa; from 3.5 to 4.0 MPa; from 4.0 to 4.5 MPa; from 4.5 to 5.0 MPa; from 5.0 to 5.5 MPa; from 5.5 to 6.0 MPa; from 6.0 to 6.5 MPa; from 6.5 to 7.0 MPa; from 7.0 to 7.5 MPa; from 7.5 to 8.0 MPa.

According to a particular embodiment, the invention relates to a method as defined above, in which, in reaction step B, the solvent is selected from ethanol, methanol and dioxane, preferably ethanol or methanol.

According to a particular embodiment, the invention relates to a method as defined above, in which, in reaction step B, the reaction medium 2 is pressurised from 1.0 to 8.0 MPa, in particular 5 MPa.

According to a particular embodiment, the invention relates to a method as defined above, in which in reaction step B, the reaction medium 2 is heated to a temperature from 100 to 250° C., in particular 200 or 220° C., preferably during from 5 to 24 hours, more preferably during 8 or 16 hours.

The expression “from 100 to 250° C.” corresponds to the following ranges: from 100 to 120° C.; from 120 to 140° C.; from 140 to 160° C.; from 160 to 180° C.; from 180 to 200° C.; from 200 to 220° C.; from 220 to 250° C.

The expression “from 5 to 24 hours” corresponds to the following ranges: from 5 to 8 hours; from 8 to 12 hours; from 12 to 16 hours; from 16 to 20 hours; from 20 to 24 hours.

### Catalyst Reuse

Reaction step B of the method of the invention is advantageously carried out without additives in the reaction medium.

Advantageously, the catalyst recovered after reaction step B of the invention is not degraded and is stable and in particular free from additives. It can advantageously be reused in another catalysed reaction step as a catalyst. It is therefore possible to repeat reaction step B according to the invention several times with the recovered catalyst or to use the catalyst recovered in reaction step B to carry out a reaction step A.

According to a particular embodiment, the invention relates to a method of preparation of ethylene glycol, as defined above, in which the catalyst is recovered at the end of a reaction step B and is reused as catalyst in another catalysed reaction step.

According to a particular embodiment, the invention relates to a method of preparation of ethylene glycol, as defined above, in which the catalyst is recovered at the end of a reaction step B and is reused as catalyst in another subsequent reaction step B.

Advantageously, the catalyst recovered at the end of a reaction step B can be used during several successive cycles of reaction steps B.

According to a particular embodiment, the invention relates to a method of preparation of ethylene glycol, as defined above, in which the catalyst is recovered at the end of a reaction step B and is reused as catalyst in a reaction step A.

According to a particular embodiment, the invention relates to a method of preparation of ethylene glycol as defined above, in which the catalyst used in reaction step A is a catalyst recovered at the end of a reaction step B.

According to a particular embodiment, the invention relates to method of preparation of ethylene glycol as defined above, in which the catalyst is recovered at the end of reaction step A and is reused as catalyst in reaction step B.

According to a particular embodiment, the invention relates to a method of preparation of ethylene glycol as defined above, in which the catalyst used in reaction step B is a catalyst recovered at the end of the reaction step A.

According to a particular embodiment, the invention relates to a method of preparation of ethylene glycol, as defined above, in which the catalyst is recovered at the end of a reaction step A and is reused as catalyst in a subsequent reaction step A.

Advantageously, the catalyst recovered at the end of a first reaction step A can be used during several successive cycles of reaction steps A.

### Flow Process

According to a particular embodiment, the invention relates to a method of preparation as defined above, in which at least one of the reaction steps of the method is carried out in continuous flow, the catalyst being a heterogeneous Pd-M/Support catalyst according to the invention.

According to a particular embodiment, the invention relates to a method of preparation of ethylene glycol as defined above, at least one of the reaction steps being carried out in continuous flow,
in which the catalyst is a heterogeneous Pd-M/Support catalyst according to the invention introduced into a column or a cartridge,
or in which the catalyst is suspended in the reaction mixture.

By way of a non-limiting example, the continuous flow process is carried out in a reactor of the following type:
a continuously stirred tank reactor (CSTR),
a flow reactor or a tubular reactor,
a reactor with a fixed or packed bed.

By way of example, the process according to the invention can be implemented in a flow chemistry apparatus, for example in commercial reactors such as “H-Cube Pro®” or “Phoenix®” from ThalesNano INC. (7 Zahony Street, Graphisoft Park, Building D, H-1031 Budapest, Hungary) or such as the “E-Series” or “R-Series flow chemistry systems” from Vapourtec Ltd (Unit 21/Park Farm Business Centre/Fornham Pk, Bury Saint Edmunds IP28 6TS, United Kingdom).

Advantageously, the continuous flow process is carried out at a temperature of 25° C. to 200° C.

Advantageously, the continuous flow process is carried out at a pressure from 0.1 MPa to 15 MPa, in particular from 0.1 to 4 MPa.

The expression “from 0.1 to 4 MPa” corresponds to the following ranges: from 0.1 to 0.5 MPa; from 0.5 to 1.0 MPa; from 1.0 to 1.5 MPa; from 1.5 to 2.0 MPa; from 2.0 to 2.5 MPa; from 2.5 to 3.0 MPa; from 3.0 to 3.5 MPa; from 3.5 to 4.0 MPa.

In a particular embodiment, the continuous flow process is carried out in a reactor in which the gases represent from 10 to 90% of the reactor volume.

The expression “from 10 to 90%” corresponds to the following ranges: from 10 to 20%; from 20 to 30%; from 30 to 40%; from 40 to 50%; from 50 to 60%; from 60 to 70%; from 70 to 80%; from 80 to 90%.

According to a particular embodiment, the continuous flow process is carried out by means allowing a contact time between the reagents from 1 second to 2 hours, in particular from 1 second to 2 minutes.

The expression “from 1 second to 2 hours” corresponds to the following ranges: from 1 to 15 seconds; from 15 to 30 seconds; from 30 seconds to 1 minute; from 1 to 2 minutes; from 2 to 15 minutes; from 15 to 30 minutes; from 30 minutes to 1 hour; from 1 to 2 hours.

According to a particular embodiment, the carbonylation reaction step A of the method is carried out in continuous flow and comprises means for introducing into the reactor the flow of CO in contact with the substrate (the alcohol) and the flow of oxygen individually or as a mixture.

According to a particular embodiment, the hydrogenation reaction step B of the method is carried out in continuous flow and comprises means for introducing into the reactor the flow of hydrogen in contact with the substrate (oxalate).

According to a particular embodiment, the carbonylation reaction step A of the method and the hydrogenation reaction step B of the method according to the invention as defined above are carried out in continuous flow and comprise means for introducing the gas flow in contact with the substrate into the reactor.

According to a particular embodiment, the carbonylation reaction step A of the process and the hydrogenation reaction step B of the method according to the invention as defined above are carried out in the same reactor.

### Catalytic Activity

The activity of the catalyst according to the invention can be described in terms of “Number of Catalytic Cycles (NCC)”.

The “Number of Catalytic Cycles (NCC)” is defined as the ratio between the number of moles of product formed (n_{prod}) and the number of moles of active catalyst species (n_{cat}).

In the case of the carbonylation reaction, the NCC is defined as the ratio between the number of moles of product formed (n_{prod}) and the number of moles of palladium in the catalyst (n_{cat}).

In the case of the hydrogenation reaction, the NCC is defined as the ratio between the number of moles of product formed (n_{prod}) and the number of moles of copper in the catalyst (n_{cat}).

The NCC is calculated as follows:

$$NCC = \frac{{n}_{prod}}{{n}_{cat}}$$

Unlike the Turnover Number (TON), which represents the maximum number of catalytic cycles that a catalyst can achieve before its total and irreversible degradation, the Catalytic Cycle Number represents the total number of catalytic cycles achieved by the catalyst under given reaction conditions. At the end of the reaction, the catalyst used would not necessarily be degraded and could therefore be reused. The NCC is therefore not a measure of the lifetime of a catalyst, but a measure of the productivity of the catalyst under the given conditions of the catalysed reaction.

### Yield

The chemical yield indicates the efficiency of the chemical reaction under study. The yield is the ratio between the quantity of product obtained and the maximum quantity that would be obtained if the reaction were complete.

The yield of reaction step B for hydrogenating oxalate to ethylene glycol according to the invention is determined as a percentage of moles of ethylene glycol obtained per mole of oxalate.

The yield of reaction step B for hydrogenating oxalate to ethylene glycol, can be determined using gas chromatography-mass spectrometry (GC-MS), in which mesitylene is used as an internal standard.

Yield can also be assessed by determining the quantity of product after purification to isolate the product.

According to a particular embodiment, the invention relates to the use as defined above, wherein said hydrogenation reaction step B has a yield of more than 70%, preferably more than 75%, preferentially more than 90%.

### Selectivity

The selectivity of a chemical reaction specifies the quantity of the desired product formed in relation to the number of moles consumed of the limiting reagent. It indicates whether several reactions are occurring in parallel, leading to unwanted by-products, or whether the reaction being carried out is the only one to consume reagent.

In the case of reaction step B, the selectivity is defined as the quantity of ethylene glycol obtained relative to the total quantity of products obtained, comprising ethylene glycol and the secondary by-products resulting from the conversion of the oxalate compound.

According to a particular embodiment, the invention relates to a method as defined above, wherein said method for preparing ethylene glycol is selective, with a selectivity of more than 70%, preferably more than 75%, more preferably more than 90%.

The expression “selective method of preparation” refers to a method enabling the product in question, ethylene glycol, to be obtained with a selectivity of more than 50%.

The expression “more than 70%” corresponds to the following ranges: more than 70%; more than 80%; more than 90%.

The expression “more than 75%” corresponds to the following ranges: more than 75%; more than 80%; more than 85%; more than 90%; more than 95%.

The expression “more than 90%” corresponds to the following ranges: more than 90%; more than 91%; more than 92%; more than 93%; more than 94%; more than 95%; more than 96%; more than 97%; more than 98%; more than 99%.

According to a particular embodiment, the invention relates to a method as defined above, in which the first catalysed reaction step A of oxidative carbonylation, from an alcohol, carbon monoxide and an oxidant, in particular molecular oxygen, optionally in the presence of a promoter, to form an oxalate compound as reaction intermediate, is selective, with a selectivity of more than 70%, preferably more than 75%, more preferably more than 90%.

The expression “selective oxidative carbonylation reaction step” refers to a carbonylation reaction step that makes it possible to obtain the target product, the oxalate compound, with a selectivity of more than 70%.

According to a particular embodiment, the invention relates to the method as defined above, in which the second catalysed reaction step B of hydrogenation of the said oxalate compound by hydrogen to obtain ethylene glycol is selective, with a selectivity of more than 70%, preferably more than 75%, preferentially more than 90%.

The expression “selective hydrogenation reaction step” refers to a hydrogenation reaction step that makes it possible to obtain the target product, ethylene glycol, with a selectivity of more than 70%.

According to a particular embodiment, the invention relates to a method as defined above, in which the first catalysed reaction step A of oxidative carbonylation, starting from an alcohol, carbon monoxide and an oxidant, in particular molecular oxygen, optionally in the presence of a promoter, to form an oxalate compound as reaction intermediate, and the second catalysed reaction step B of hydrogenation of the said oxalate compound by hydrogen to obtain ethylene glycol, are selective, with a selectivity of more than 70%, preferably more than 75%, preferentially more than 90%.

The expression “selective reaction step” refers to a reaction step that makes it possible to obtain the target product, the oxalate compound or the ethylene glycol, with a selectivity of more than 70%.

According to a particular embodiment, the invention relates to a method as defined above, in which:
reaction step A comprises
   bringing into contact:
      an alcohol selected from methanol or ethanol
      carbon monoxide, in particular at 6.5 MPa,
      molecular oxygen, in particular at 1.5 MPa,
      a catalyst of formula Pd-M/Support in which M represents Cu or Ag,
      tetramethylammonium iodide as a promoter,
      triethylamine as a base,
      acetonitrile as solvent,
   to obtain a reaction medium 1 which may be pressurised from 0.1 to 15 MPa, in particular to 8.0 MPa
      heating said reaction medium, in particular to a temperature of about 90° C., preferably for 16 hours,
to obtain the oxalate compound;
reaction step B comprises
   bringing into contact:
      said oxalate compound
      dihydrogen, in particular at 5.0 MPa,
      said catalyst of formula Pd-M/Support in which M represents Cu or Ag,
      ethanol as a solvent,
to obtain a reaction medium 2 which may be pressurised from 0.1 to 15 MPa, in particular 5.0 MPa,
heating said reaction medium, in particular to a temperature of about 200° C., preferably for 16 hours, or to a temperature of about 220° C., preferably for 8 hours,
   to obtain ethylene glycol.

### Pd—Cu/ZrO₂ Catalyst

Another object of the invention concerns a bimetallic catalyst of palladium and copper on a zirconium dioxide support of formula Pd—Cu/ZrO₂, comprising:
a palladium content from 0.1 to 10%, in particular 2%, and a copper content from 0.1 to 40%, in particular 10%, by weight relative to the total weight of the catalyst and,
a surface area, measured by BET, from 1 to 50 m2/g, in particular from 1 to 10 m²/g, preferably from 5 to 7 m2/g.

The inventors have unexpectedly observed that bimetallic catalysts of palladium and copper on a zirconium dioxide support with a low specific surface area of approximately 5 m²/g are more efficient than a catalyst with a higher specific surface area of 63 m2/g or 81 m2/g. This efficiency applies to both the carbonylation reaction (step A) and the hydrogenation reaction (step B) for the preparation of ethylene glycol, making it possible in particular for the hydrogenation reaction to achieve a yield of 92% and a selectivity of 94%, as revealed in the results of example 24. In fact, it would be expected that a specific surface area greater than 50 m2/g would allow greater accessibility to the substrates and favour the yields of the catalyst.

In a particular embodiment, the invention relates to a bimetallic catalyst of palladium and copper on a zirconium dioxide support of formula Pd—Cu/ZrO₂, as defined above further comprising:
a crystalline phase of zirconium dioxide, analysed by X-ray diffraction, crystallised in monoclinic baddeleyite comprising a crystallite size from 20 to 100 nm, preferably from 20 to 50 nm, and optionally comprising hafnium atoms as an impurity.

For the purposes of this invention, by “baddeleyite” it is meant a natural zirconium oxide of formula ZrO₂, containing from 0.1% to 5% hafnium oxide, and crystallising in a monoclinic crystal system. The characteristics of baddeleyite, such as its composition and crystallographic structure, in particular the space group with the dimensions of the crystal lattice, are reported and available in the prior art and are known to those skilled in the art, such as Kudoh, Y. et al, (Phys Chem Minerals 13, 233-237 (1986)) or Mccullough J D. et al (Acta Crystallographica 12 (1959) 507-511).

In a particular embodiment, the crystalline phase of zirconium dioxide comprises impurities such as Hafnium (Hf) atoms.

From 20 to 100 nm refers to the following ranges: from 20 to 30 nm; from 30 to 40 nm; from 40 to 50 nm; from 50 to 60 nm; from 60 to 70 nm; from 70 to 80 nm; from 80 to 90 nm; from 90 to 100 nm.

In another particular embodiment, the invention concerns a bimetallic catalyst of palladium and copper on a zirconium dioxide support of formula Pd—Cu/ZrO₂, as defined above, in the form of two populations of particles:
a first population of particles with a polyhedral-type morphology and
a second population of particles smaller in size than the first population and having a rounded and entangled morphology, with sizes from 10 nm to 1 micrometre, said particles forming clusters from 1 to 100 micrometres.

For the purposes of this invention, by “polyhedral-type particles” it is meant particles with edges, corners or bevelled edges.

For the purposes of this invention, by “particles with a rounded morphology” it is meant particles that do not have an edge, corner or a bevelled edge.

For the purposes of this invention, “entangled particles” meant rounded particles agglomerated together or having a fused appearance, forming a visible surface of protuberances,

For the purposes of this invention, by “particle cluster” it is meant a set of entangled particles that are either visually or mechanically coherent with one another.

In a particular embodiment, the invention relates to a bimetallic catalyst of palladium and copper on a zirconium dioxide support of formula Pd—Cu/ZrO₂, as defined above, further comprising:
copper atoms in oxidation state (I) and (II), and
palladium atoms in oxidation state (II).

Advantageously, the molar quantity of copper atoms in an oxidation state (I) is greater than that of copper atoms in an oxidation state (II).

Pd—Ag/γ-Al₂O₃ Catalyst

Another object of the invention relates to a bimetallic catalyst of palladium and silver on an alumina support of formula Pd—Ag/γ-Al₂O₃, comprising:
a palladium content from 0.1 to 10%, in particular 2%, and a silver content from 0.1 to 40%, in particular 15%, by weight relative to the total weight of the catalyst,
silver atoms in oxidation states (I) and (II) and palladium atoms in oxidation state (II),
   said catalyst being in the form of a population of agglomerated, generally spherical particles from 10 to 100 micrometres in size having a porous morphology with a honeycomb appearance on the surface.

For the purposes of this invention, “porous morphology with a honeycomb appearance” means a visible morphology of a set of craters with common edges in the form of ridges.

Pd—Cu/γ-Al₂O₃ catalyst

Another object of the invention relates to a bimetallic catalyst of palladium and copper on an alumina support of formula Pd—Cu/γ-Al₂O₃ catalyst, comprising:
a palladium content from 0.1 to 10%, in particular 2%, and a copper content from 0.1 to 40%, in particular 10%, by weight relative to the total weight of the catalyst,
copper atoms in oxidation state (II) and palladium atoms in oxidation state (II).
   said catalyst being in the form of a population of globally spherical, agglomerated particles from 10 to 100 micrometres in size, having a porous morphology with a honeycomb appearance on the surface.

### Method of Preparation of the Catalyst

Another object of the invention relates to a method of preparation of a catalyst of formula Pd-M/Support, comprising palladium and a metal M on an oxide support, in which M represents Cu or Ag, comprising:
a step C of impregnating a palladium salt and a copper or silver salt, dissolved in an aqueous solution, in particular in a volume of water from 5 to 10 mL, on an oxide support in powder form, with a ratio solution mass/support mass from 0.6 to 1.0;
to obtain the Pd-M/Support catalyst in the form of a homogeneous material,
in particular said step C comprising:
   the use of a palladium salt concentration calculated to obtain a palladium content from 0.1 to 10% by weight relative to the total weight of the catalyst,
   the use of a copper or silver salt concentration calculated to obtain a copper or silver content from 0.1 to 40% by weight, based on the total weight of the catalyst,
a step D of drying said homogeneous material, in particular at a temperature from 60 to 100° C., in particular 80° C., preferably for a period from 10 to 24 hours, in particular 16 hours, in order to obtain the Pd-M/support catalyst in the form of an anhydrous homogeneous material,
an activation step E, in particular comprising calcination of said anhydrous homogeneous material at a temperature from 200 to 1000° C., in particular 600° C., preferably for a period from 1 to 15 hours, in particular 2 hours, in order to obtain said catalyst.

Advantageously, the palladium salt is palladium nitrate and the copper or silver salt is copper or silver nitrate.

The inventors have surprisingly found that the method of preparation makes it possible to obtain active and effective catalysts for the two reactions of carbonylation of alcohol to oxalate and hydrogenation of oxalate to ethylene glycol in the ethylene glycol preparation process, without the need for a preliminary step of reducing the metal atoms Pd, Cu or Ag under a flow of hydrogen.

In a particular embodiment, the invention relates to a method of preparation of a Pd—Cu/ZrO₂ catalyst according to the catalyst of the invention as defined above, comprising:
a step C of impregnating a palladium salt and a copper salt, in particular palladium nitrate and copper nitrate, dissolved in an aqueous solution, in particular in a volume of water from 5 to 10 mL, on a zirconium dioxide support, in particular in powder form, with a ratio of solution mass/support mass from 0.6 to 1.0;
to obtain Pd—Cu/ZrO₂ catalyst in the form of a homogeneous material, in particular said step C comprises:
   the use of a palladium salt concentration calculated to obtain a palladium content from 0.1 to 10% by weight relative to the total weight of the catalyst,
   the use of a copper salt concentration calculated to obtain a copper content from 0.1 to 40% by weight relative to the total weight of the catalyst,
a step D of drying said homogeneous material, in particular at a temperature from 60 to 100° C., in particular 80° C., preferably for a period from 10 to 24 hours, in particular 16 hours, to obtain Pd—Cu/ZrO₂ catalyst in the form of an anhydrous homogeneous material,
an activation step E, in particular comprising calcination of said anhydrous homogeneous material at a temperature from 200 to 1000° C., in particular 600° C., preferably for a period from 1 to 15 hours, in particular 2 hours, in order to obtain said catalyst.

Advantageously, said support used to prepare the catalyst is a zirconium dioxide with a specific surface area from 1 to 50 m2/g, in particular from 1 to 10 m²/g, preferably from 5 to 7 m2/g.

Advantageously, said support used for preparing the catalyst is a zirconium dioxide crystallised in monoclinic baddeleyite and comprises a crystallite size from 20 to 100 nm, preferably from 20 to 50 nm, and a specific surface area from 1 to 50 m2/g, in particular from 1 to 10 m²/g, preferably from 5 to 7 m2/g, and optionally comprises hafnium atoms as impurities.

In a particular embodiment, the invention relates to a method of preparation of a Pd—Ag/γ-Al₂O₃ catalyst, comprising:
a step C of impregnating a palladium salt and a silver salt, in particular palladium nitrate and copper nitrate, dissolved in an aqueous solution, in particular in a volume of water from 5 to 10 mL, on an alumina support (γ-Al₂O₃), in particular in powder form, with a ratio of solution mass/support mass from 0.6 and 1.0;
to obtain Pd—Ag/v-Al₂O₃ catalyst in the form of a homogeneous material,
in particular said step C comprising:
   the use of a palladium salt concentration calculated to obtain a palladium content from 0.1 to 10% by weight relative to the total weight of the catalyst,
   the use of a silver salt concentration calculated to obtain a silver content from 0.1 to 40% by weight relative to the total weight of the catalyst,
a step D of drying said homogeneous material, in particular at a temperature from 60 to 100° C., in particular 80° C., preferably for a period from 10 to 24 hours, in particular 16 hours, in order to obtain Pd—Ag/γ-Al₂O₃ catalyst in the form of an anhydrous homogeneous material,
an activation step E, in particular comprising calcination of said anhydrous homogeneous material at a temperature of 200 to 1000° C., in particular 600° C., preferably for a period from 1 to 15 hours, in particular 2 hours, in order to obtain said catalyst.

### Pd-M Catalyst/Support Prepared According to the Invention

Another object of the invention relates to a supported bimetallic catalyst of formula Pd-M/Support, comprising palladium and a metal M on a support, in which M represents Cu or Ag, obtainable by a method of preparation of a catalyst as defined above.

### Pd—Cu/ZrO₂ Catalyst Prepared According to the Invention

In a particular embodiment, the invention relates to a bimetallic catalyst of palladium and copper on a zirconium dioxide support of formula Pd—Cu/ZrO₂, obtainable by a method of preparation of a catalyst as defined above.

According to one embodiment, the invention relates to a bimetallic catalyst of palladium and copper on a zirconium dioxide support of formula Pd—Cu/ZrO₂, obtainable by a preparation process comprising:
a step C of impregnating a palladium salt and a copper salt, in particular palladium nitrate and copper nitrate, dissolved in an aqueous solution, in particular in a volume of water from 5 to 10 mL, on a zirconium dioxide support, in particular in powder form, with a ratio of solution mass/support mass from 0.6 to 1.0;
to obtain Pd—Cu/ZrO₂ catalyst in the form of a homogeneous material,
in particular said step C comprises:
   the use of a palladium salt concentration calculated to obtain a palladium content from 0.1 to 10% by weight relative to the total weight of the catalyst,
   the use of a copper salt concentration calculated to obtain a copper content from 0.1 to 40% by weight relative to the total weight of the catalyst,
a step D of drying said homogeneous material, in particular at a temperature from 60 to 100° C., in particular 80° C., preferably for a period from 10 to 24 hours, in particular 16 hours, to obtain Pd—Cu/ZrO₂ catalyst in the form of an anhydrous homogeneous material,
an activation step E, in particular comprising calcination of said anhydrous homogeneous material at a temperature from 200 to 1000° C., in particular 600° C., preferably for a period from 1 to 15 hours, in particular 2 hours, in order to obtain said catalyst.

In a particular embodiment, the invention relates to a bimetallic catalyst of palladium and silver on an alumina support (γ-Al₂O₃) of formula Pd—Ag/γ-Al₂O₃, obtainable by a method of preparation of a catalyst as defined above.

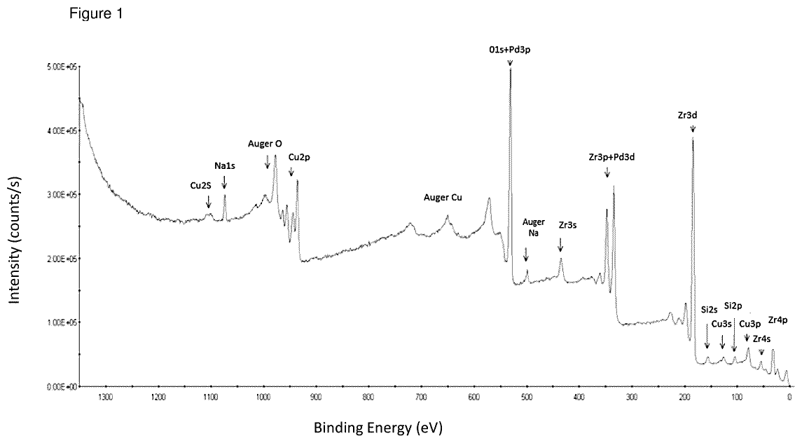

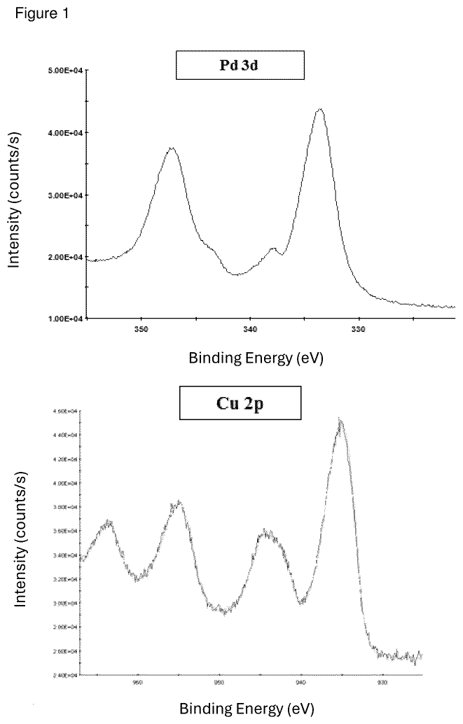

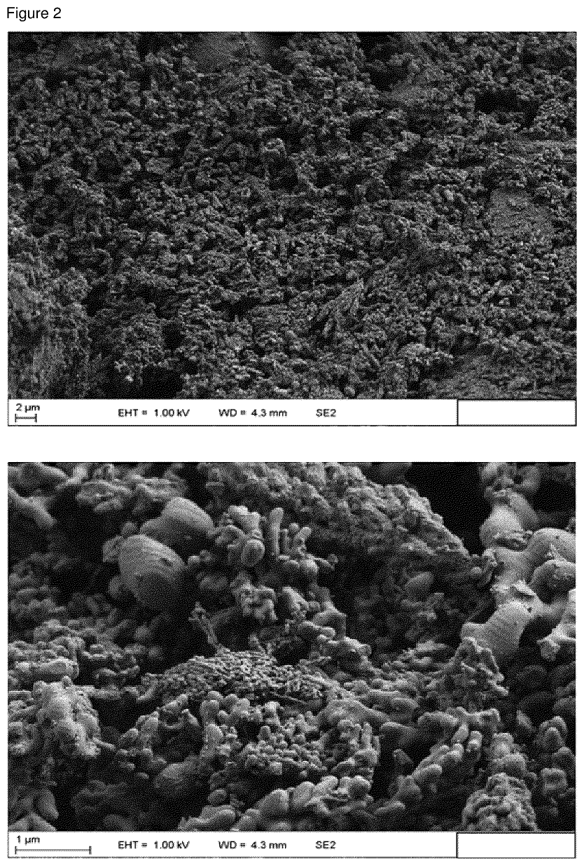

Another object of the invention concerns a bimetallic catalyst of palladium and copper on a zirconium dioxide support of formula Pd—Cu/ZrO₂, comprising:
palladium content from 0.1 to 10%, in particular 2%, and a copper content from 0.1 to 40%, in particular 10%, by weight relative to the total weight of the catalyst;
in which
   said catalyst has an X-ray photoelectron spectrometry spectrum similar to that shown in FIG. 1 for a composition of 2% Pd and of 10% Cu by weight relative to the total weight of the catalyst;
   and said catalyst has a morphological appearance by scanning microscopy analysis similar to FIG. 2 for a composition of 2% Pd and 10% Cu by weight relative to the total weight of the catalyst.

The following examples and figures illustrate the invention, without limiting its scope.

FIG. 1 shows the X-ray photoelectron spectrometry spectrum of a Pd—Cu/ZrO₂ catalyst with a composition of 2% Pd and 10% Cu by weight relative to the total weight of the catalyst, prepared using a zirconium dioxide support with a specific surface area from 5 to 7 m²/g.

FIG. 2 shows two scanning electron microscopy images of a Pd—Cu/ZrO₂ catalyst with a composition of 2% Pd and 10% Cu by weight relative to the total weight of the catalyst, prepared with a zirconium dioxide support with a specific surface area from 5 to 7 m²/g.

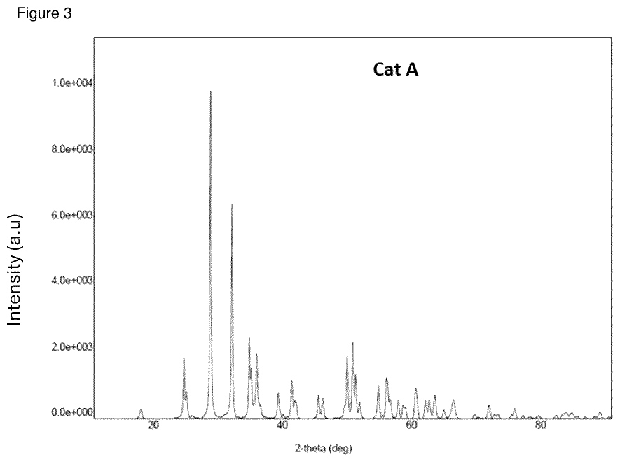

FIG. 3 shows the diffraction pattern of a Pd(2%)Cu(10%)/ZrO₂ catalyst prepared with a zirconium dioxide support with a specific surface area from 5 to 7 m²/g.

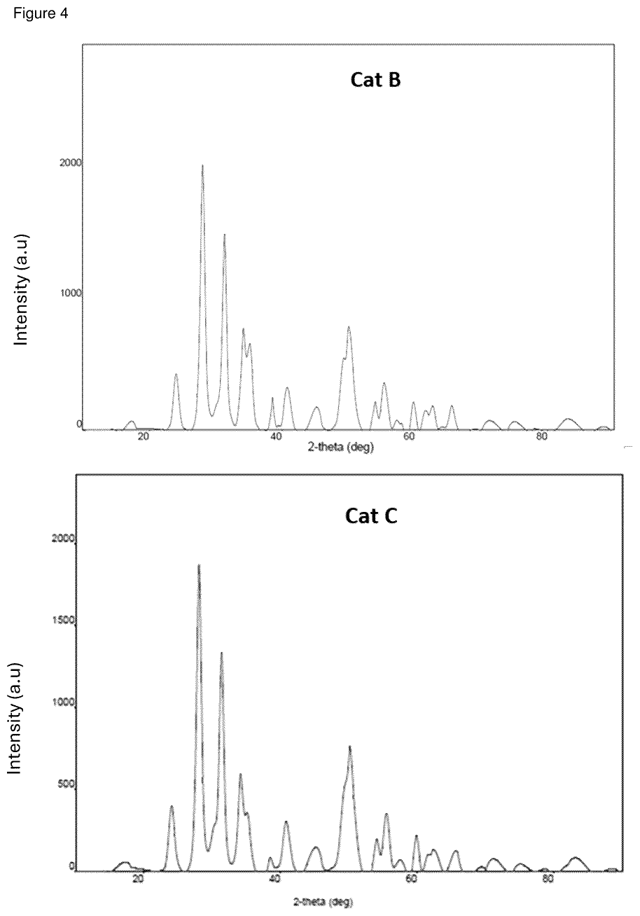

FIG. 4 shows the diffraction patterns of two catalysts, Pd(2%)Cu(10%)/ZrO₂ and Pd(1%)Cu(3%)/ZrO₂, prepared using zirconium dioxide with a specific surface area greater than 85 m²/g.

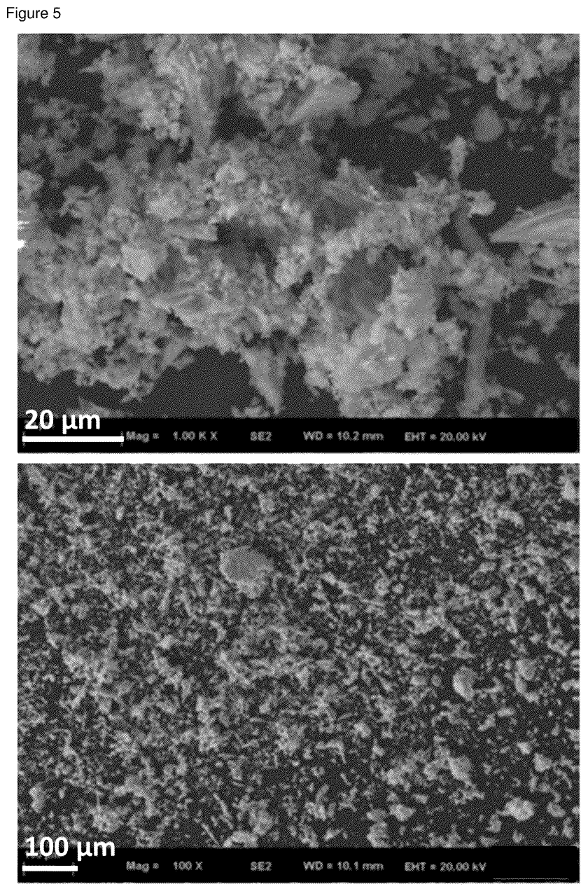

FIG. 5 shows SEM images of a Pd(2%)Cu(10%)/ZrO₂ catalyst prepared using zirconium dioxide with a specific surface area from 5 to 7 m²/g.

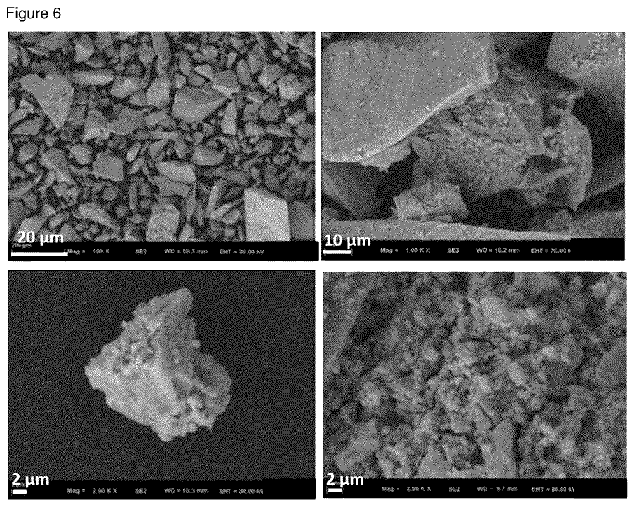

FIG. 6 shows SEM images of a Pd(2%)Cu(10%)/ZrO₂ catalyst prepared using zirconium dioxide with a specific surface area greater than 85 m²/g.

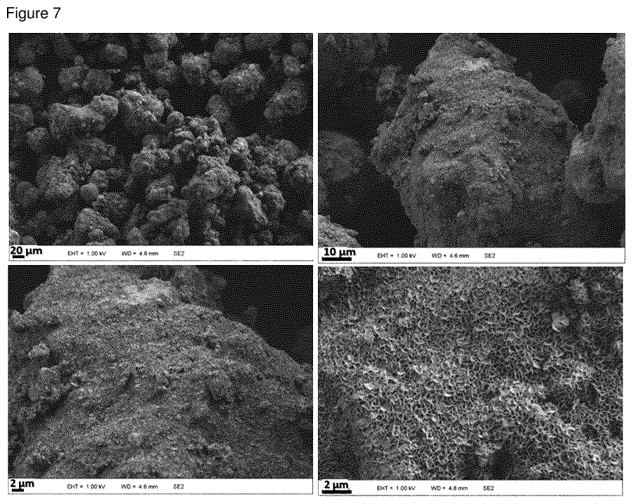

FIG. 7 shows SEM images of a Pd(2%)Cu(10%)/γ-Al₂O₃ catalyst

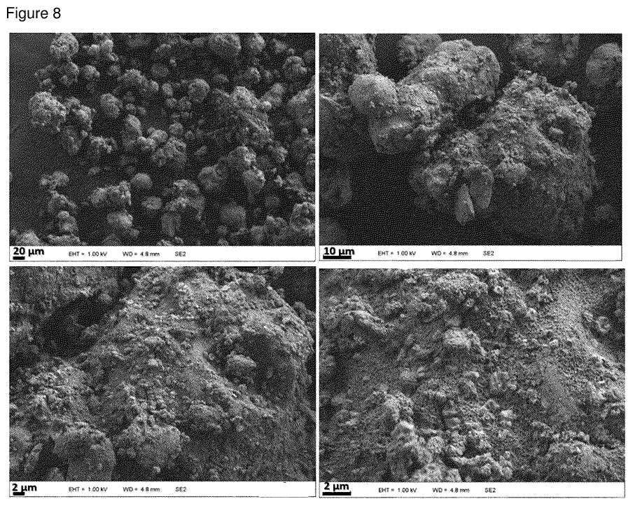

FIG. 8 shows SEM images of a Pd(2%)Ag(15%)/γ-Al₂O₃ catalyst

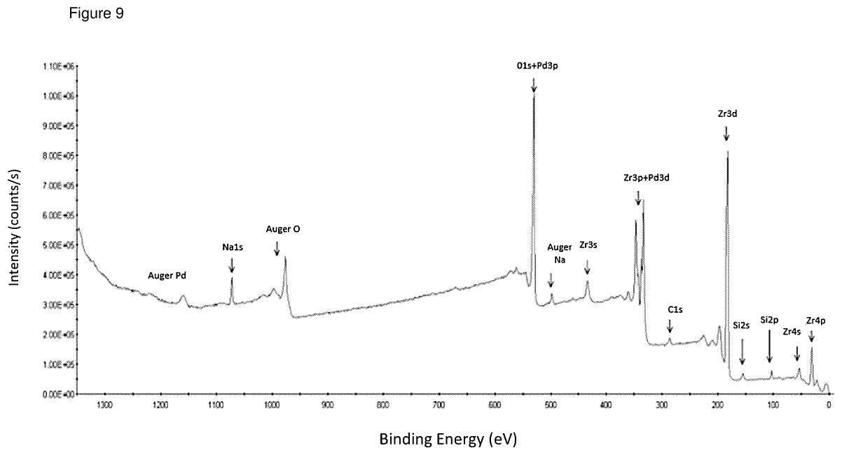

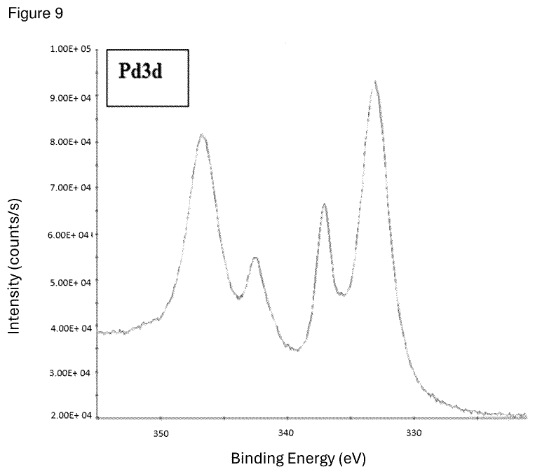

FIG. 9 shows the XPS spectra of a Pd(2%)/ZrO₂ catalyst.

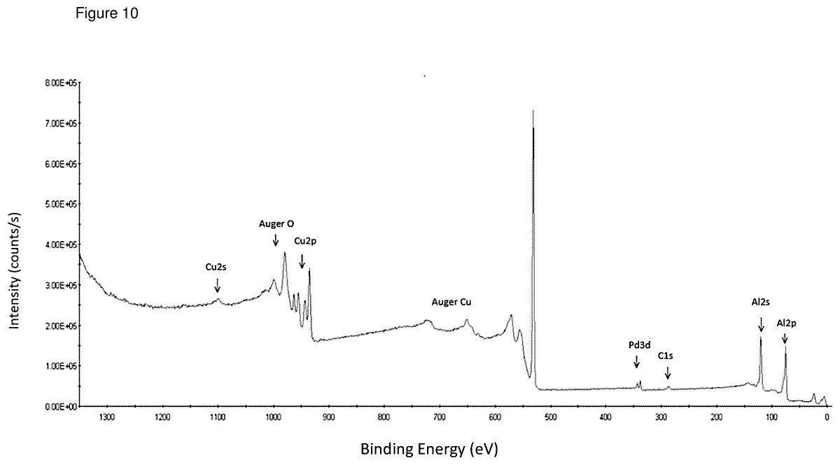

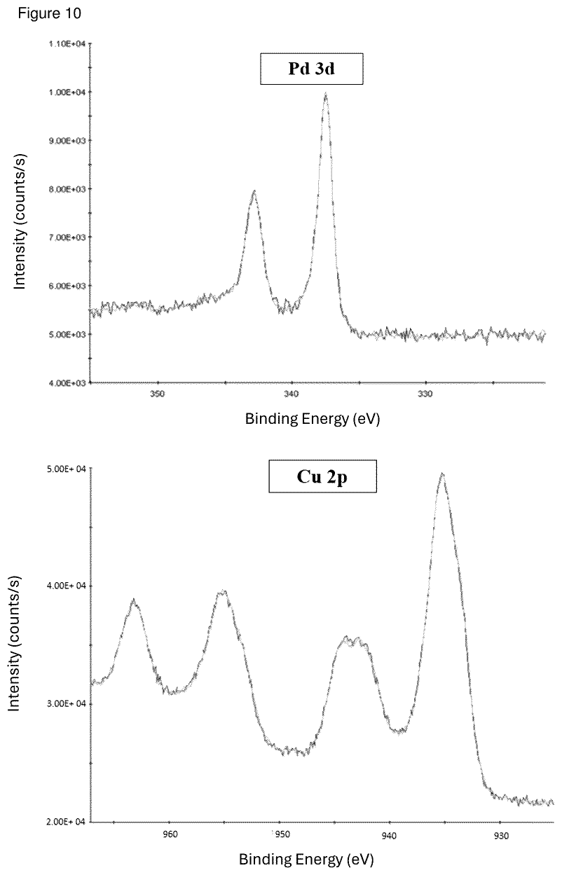

FIG. 10 shows the XPS spectra of a Pd(2%)Cu(10%)/γ-Al₂O₃ catalyst.

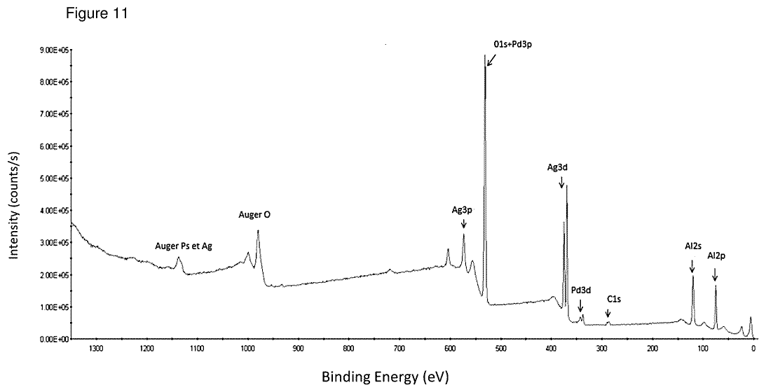

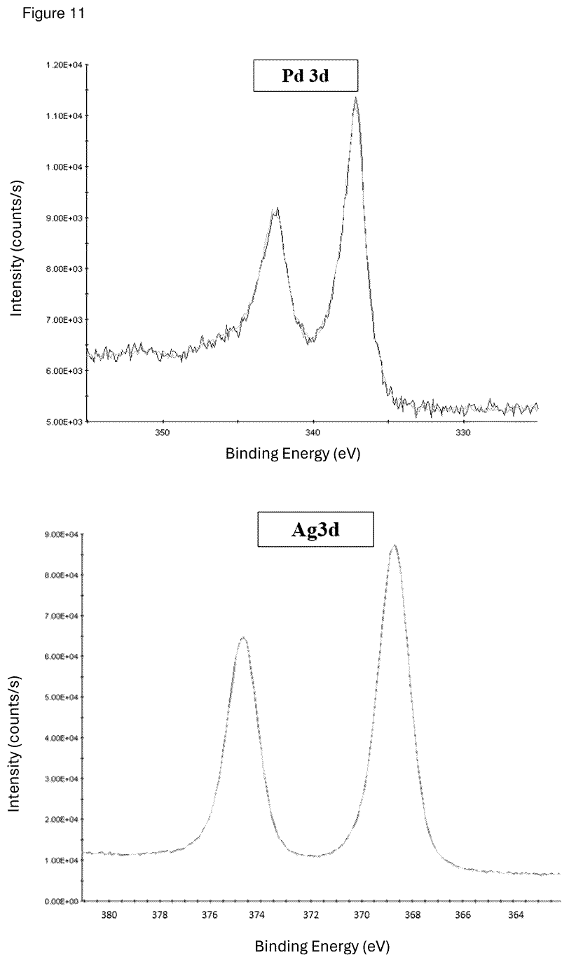

FIG. 11 shows the XPS spectra of a Pd(2%)Ag(15%)/γ-Al₂O₃ catalyst.

### EXAMPLES

### Example 1—Materials and Methods

The ZrO₂ support, with a specific surface area of 5 to 7 m²/g, is supplied by Sterm Chemicals (15 Rue de l'Atome, 67800 Bischheim) (product number 93-4013. Without further clarification, hereafter the support named ZrO₂ is that supplied by Sterm Chemicals.

The ZrO₂ support (monoclinic phase), with a specific surface area greater than 85 m²/g is supplied by Alfa Aesar (Thermo Fisher Scientific) with product number AA4381522.

The γ-Al₂O₃ support is supplied by Sterm Chemicals (15 Rue de l′Atome, 67800 Bischheim) with reference number 13-2525.

The SiO₂ support (40-63 μm) was supplied by VWR chemicals, reference number 151125P. Palladium nitrate (Pd(NO₃)₂·xH₂O) and other metal salts such as Cu(NO₃)₂·3H₂O, AgNO₃ were supplied by Fischer.

The 450 mL and 1 L autoclaves are supplied by Parr Instrument Company.

### Example 2—General Procedure for the Preparation of Heterogeneous Pd/ZrO₂ Catalysts

Pd(NO₃)₂·xH₂O was dissolved in a minimum volume of demineralised water, between 5 and 10 mL, forming a solution. This solution containing the metal precursors was added to the appropriate amount of zirconium dioxide support and the resulting paste of ZrO₂ was mixed at room temperature until a homogeneous material was obtained. The material was then dried at 80° C. for 16 hours and calcined at 600° C. for 2 hours to obtain the catalyst.

### Example 3—General Procedure for the Preparation of Heterogeneous Cu/ZrO₂ Catalysts

Cu(NO₃)₂·3H₂O was dissolved in a minimum volume of demineralised water, between 5 and 10 mL, forming a solution. This solution containing the metal precursors was added to the appropriate amount of zirconium dioxide support and the resulting paste of ZrO₂ was mixed at room temperature until a homogeneous material was obtained. The material was then dried at 80° C. for 16 hours and calcined at 600° C. for 2 hours to obtain the catalyst.

### Example 4—General Procedure for the Preparation of Heterogeneous Pd—Cu/ZrO₂ Catalysts

Pd(NO₃)₂·xH₂O and Cu(NO₃)₂·3H₂O were dissolved in a minimum volume of demineralised water, between 5 and 10 mL, forming a solution. This solution containing the metal precursors was added to the appropriate amount of zirconium dioxide support and the resulting paste of ZrO₂ was mixed at room temperature until a homogeneous material was obtained. The material was then dried at 80° C. for 16 hours and calcined at 600° C. for 2 hours to obtain the catalyst.

### Example 5—Preparation of Heterogeneous Mono- and Bimetallic Pd/ZrO₂, Cu/ZrO₂, Pd—Cu/ZrO₂ Catalysts

Table 1 below reports the preparation conditions for the Pd/ZrO₂, Cu/ZrO₂ and Pd—Cu/ZrO₂ catalysts prepared according to examples 2, 3 and 4.

TABLE 1
Prepared Pd/ZrO₂, Cu/ZrO₂ and Pd—Cu/ZrO₂ catalysts.

| | Support | | |
|---|---|---|---|
| | ZrO₂ | Pd(NO₃)₂•xH₂O | Cu(NO₃)₂•3H₂O |
| Catalyst | (g) | (mg) | (mg) |
| Pd(2%)/ZrO₂ | (10 g) | 504 mg | |
| Cu(10%)/ZrO₂ | (10 g) | | 3802 |
| Pd (2%)—Cu(10%)/ZrO₂ | (10 g) | 504 mg | 3802 |

### Example 6—General Procedure for the Preparation of Heterogeneous Pd—Cu/γ-Al₂O₃ Catalysts

Pd(NO₃)₂·xH₂O and Cu(NO₃)₂·3H₂O were dissolved in a minimum volume of demineralised water, between 5 and 10 mL, forming a solution. This solution containing the metal precursors was added to the appropriate amount of γ-Al₂O₃ support and the paste obtained was mixed at room temperature until a homogeneous material was obtained. The material was then dried at 80° C. for 16 hours and calcined at 600° C. for 2 hours to obtain the catalyst.

### Example 7—Preparation of Heterogeneous Bimetallic Pd—Cu/γ-Al₂O₃ Catalysts

Table 2 below reports the conditions for preparing a Pd—Cu/γ-Al₂O₃ catalyst prepared according to example 6.

TABLE 2
Prepared Pd—Cu/γ-Al₂O₃ catalyst.

| | Support | | |
|---|---|---|---|
| | ZrO₂ | Pd(NO₃)₂•xH₂O | Cu(NO₃)₂•3H₂O |
| Catalyst | (g) | (mg) | (mg) |
| Pd (2%)—Cu(10%)/ZrO₂ | (10 g) | 504 mg | 3802 |

### Example 8—General Procedure for the Preparation of Heterogeneous Ag/γ-Al₂O₃ Catalysts

AgNO₃ was dissolved in a minimum volume of demineralised water, between 5 and 10 mL, to form a solution. This solution containing the metal precursors was added to the appropriate amount of γ-Al₂O₃ support and the paste obtained was mixed at room temperature until a homogeneous material was obtained. The material was then dried at 80° C. for 16 hours and calcined at 600° C. for 2 hours to obtain the catalyst.

### Example 9—General Procedure for the Preparation of Heterogeneous Pd—Ag/γ-Al₂O₃ Catalysts

Pd(NO₃)₂·xH₂O and AgNO₃ were dissolved in a minimum volume of demineralised water, between 5 and 10 mL, forming a solution. This solution containing the metal precursors was added to the appropriate amount of support γ-Al₂O₃ and the paste obtained was mixed at room temperature until a homogeneous material was obtained. The material was then dried at 80° C. for 16 hours and calcined at 600° C. for 2 hours to obtain the catalyst.

Table 3 below reports the preparation conditions for the Ag/γ-Al₂O₃ and Pd—Ag/γ-Al₂O₃ catalysts prepared according to examples 8 and 9.

TABLE 3
Prepared Ag/γ-Al₂O₃ and Pd—Ag/γ-Al₂O₃ catalysts.

| | Support | | |
|---|---|---|---|
| | γ-Al₂O₃ | Pd(NO₃)₂•xH₂O | AgNO₃ |
| Catalyst | (g) | (mg) | (mg) |
| Ag(15%)/γ-Al₂O₃ | (10 g) | | 2362 |
| Pd (2%)—Ag(10%) / γ-Al₂O₃ | (10 g) | 504 mg | 1575 |

### Example 10—General Procedure for the Preparation of Heterogeneous Pd—Ag/SiO₂ Catalysts

Pd(NO₃)₂·xH₂O and AgNO₃ were dissolved in a minimum volume of demineralised water, between 5 and 10 mL, forming a solution. This solution containing the metal precursors was added to the appropriate amount of SiO₂ support and the paste obtained was mixed at room temperature until a homogeneous material was obtained. The material was then dried at 80° C. for 16 hours and calcined at 600° C. for 2 hours to obtain the catalyst.

Table 4 below reports the conditions for preparing a Pd—Ag/SiO₂ catalyst prepared according to example 10.

TABLE 4
Prepared Pd—Ag/SiO₂ catalyst.

| | Support | | |
|---|---|---|---|
| | ZrO₂ | Pd(NO₃)₂•xH₂O | AgNO₃ |
| Catalyst | (g) | (mg) | (mg) |
| Pd (2%)—Ag(10%) / SiO₂ | (10 g) | 504 mg | 1575 |

### Example 11—General Heterogeneous Catalysis Procedure for the Oxidative Carbonylation of Methanol to Oxalates

A heterogeneous palladium catalyst (0.24 mmol Pd), tetrabutylammonium iodide TBAI (554 mg, 1.5 mmol) as promoter, tri-ethylamine Et₃N (0.14 mL, 1.0 mmol), acetonitrile (50 mL) and methanol (25 mL) were introduced into a 450 mL Parr autoclave equipped with a magnetic stirrer. The reactor was sealed and the reaction mixture was purged three times with nitrogen (5 bar) and twice with oxygen (5 bar).

The autoclave was then pressurised with 15 bars of oxygen and a further 65 bars of carbon monoxide (total pressure of 80 bars). The reaction medium was then stirred at 90° C. for 16 h.

Once the reaction was complete, the autoclave was brought to room temperature before being depressurised and purged three times with nitrogen (5 bar).

The final mixture obtained was then filtered and transferred to a 250 mL flask.

The reaction solvent and excess alcohol were separated by evaporation on a rotary evaporator.

The diethyloxalate was recovered after purification by recrystallisation in di-ethyl ether and the isolated yields were calculated.

For reactions in which the alcohol is used as a substrate in the presence of a solvent and also for reactions carried out in the absence of a solvent in which the alcohol plays the role of both substrate and solvent, the results are described in NCC in order to assess the catalytic efficiency, particularly due to the use in excess of alcohol as a substrate.

The NCC is calculated as follows:

NCC=number of moles of product formed/number of moles of Pd

### Example 12—General Heterogeneous Catalysis Procedure for the Oxidative Carbonylation of Ethanol to Oxalates

A heterogeneous palladium catalyst (0.24 mmol Pd), tetrabutylammonium iodide TBAI (1.5 to 3.6 mmol), tri-ethylamine Et₃N (1.0 to 4.75 mmol), optionally acetonitrile MeCN (0 to 50 mL) as solvent and ethanol (25 to 75 mL) were introduced into a 450 mL Parr autoclave equipped with a magnetic stirrer. The reactor was sealed and the reaction mixture was purged three times with nitrogen (5 bar) and twice with oxygen (5 bar).

The autoclave was then pressurised with 15 bars of oxygen and a further 65 bars of carbon monoxide (total pressure of 80 bars). The reaction medium was then stirred at 90° C. for 16 h.

Once the reaction was complete, the autoclave was brought to room temperature before being depressurised and purged three times with nitrogen (5 bar).

The final mixture obtained was then filtered and transferred to a 250 mL flask.

The reaction solvent and the excess alcohol were separated by evaporation on a rotary evaporator.

The diethyloxalate was recovered after purification by vacuum distillation at 120° C./50-20 mbar and the isolated yields were calculated.

### Example 13—General Heterogeneous Catalysis Procedure for the Hydrogenation of Dialkyl Oxalates

A heterogeneous copper or silver catalyst (4 mmol Cu or Ag), dialkyloxalate (20 mmol) and ethanol (50 mL) were introduced into a 450 mL Parr autoclave equipped with a magnetic stirrer.

The reactor was sealed and the reaction mixture was purged three times with nitrogen (5 bar) and twice with hydrogen (5 bar).

The autoclave was then pressurised with 50 bars of hydrogen. The reaction medium was then stirred at 200° C. for 16 h or 220° C. for 8 h.

Once the reaction was complete, the autoclave was brought to room temperature before being depressurised and purged three times with nitrogen (5 bar).

The final mixture obtained was diluted in ethanol or methanol and then an internal standard was added (mesitylene) to calculate the yield by using GC-MS.

### Example 14—Tests Carried Out with Pd/ZrO₂ Catalyst

Tables 5 and 6 below report respectively the test conditions of oxidative carbonylation (reaction step A) and of hydrogenation (reaction step B) with a Pd/ZrO₂ catalyst.

### Reaction Step A—Carbonylation

TABLE 5
Test conditions of oxidative carbonylation with a Pd/ZrO₂ catalyst and the obtained results

| | | | | | Pressure | | | |
|---|---|---|---|---|---|---|---|---|
| | | | | Solvent | CO/O₂ | T | Time | Obtained |
| Reaction | Catalyst | additives | Substrate | (ml) | (bar) | (° C.) | (h) | weight |
| A1-1 | Pd(2%)ZrO₂ | Et₃N | 25 mL | CH₃CN | 65/15 | 90 | 16 | 4.73 g |
| | (0.24 mmol) | (1 mmol) | EtOH | 50 mL | (CO/O₂) | | | |
| | | TBAI | | | | | | |
| | | (1.5 mmol) | | | | | | |
| A1-2 | Pd(2%)ZrO₂ | Et₃N | 75 mL | | 65/15 | 90 | 16 | 3.6 g |
| | (0.24 mmol) | (1 mmol) | EtOH | | (CO/O₂) | | | |
| | | TBAI | | | | | | |
| | | (1.5 mmol) | | | | | | |

### Reaction Step B—Hydrogenation

TABLE 6
Test conditions of hydrogenation with a Pd/ZrO₂ catalyst and the obtained results.

| | | | Solvent | Pressure | T | Time | Yield |
|---|---|---|---|---|---|---|---|
| Reaction | Catalyst | Substrate | (ml) | H₂ (bar) | (° C.) | (h) | (%)* |
| B1-1 | Pd(2%)ZrO₂ | 20 mmol | EtOH | 50 (H₂) | 200 | 16 | 0 |
| | (0.47 mmol~2.3 mol %) | DEO | 50 mL | | | | |

*Hydrogenation yield is calculated using GC-MS, mesitylene is used as internal standard

### Example 15—Tests Carried Out with Cu/ZrO₂ Catalyst

Tables 7 and 8 below report respectively the test conditions of oxidative carbonylation (reaction step A) and of hydrogenation (reaction step B) with a Cu/ZrO₂ catalyst.

### Reaction Step A—Carbonylation

TABLE 7
Test conditions of oxidative carbonylation with a Cu/ZrO₂ catalyst and the obtained results.

| | | | | | Pressure | | | |
|---|---|---|---|---|---|---|---|---|
| | | | | Solvent | CO/O₂ | T | Time | |
| Reaction | Catalyst | additives | Substrate | (ml) | (bar) | (° C.) | (h) | NCC |
| A2 | Cu(10%)ZrO₂ | Et₃N | 25 mL | CH₃CN | 65/15 | 90 | 16 | 0 |
| | (4 mmol) | (1 mmol) | EtOH | 50 mL | (CO/O₂) | | | |
| | | TBAI | | | | | | |
| | | (1.5 mmol) | | | | | | |

### Reaction Step B—Hydrogenation

TABLE 8
Test conditions of hydrogenation with a Cu/ZrO₂ catalyst and the obtained results.

| | | | Solvent | Pressure | T | Temps | |
|---|---|---|---|---|---|---|---|
| Reaction | Catalyst | Substrate | (ml) | H₂ (bar) | (° C.) | (h) | Yield (%)* |
| B2 | Cu(10%)ZrO₂ | 20 mmol | EtOH | 50 (H₂) | 200 | 16 | 13 (12% |
| | (4 mmol = | DEO | 50 mL | | | | selectivity) |
| | 20 mol %) | | | | | | |

*Hydrogenation yield is calculated using GC-MS, mesitylene is used as internal standard

### Example 16—Tests Carried Out with Ag/γ-Al₂O₃ Catalyst

Tables 9 and 10 below respectively report the test conditions of oxidative carbonylation (reaction step A) and of hydrogenation (reaction step B) with a Ag/γ-Al₂O₃ catalyst.

### Reaction Step A—Carbonylation

TABLE 9
Test conditions of oxidative carbonylation with a Ag/γ-Al₂O₃ and the obtained results.

| | | | | | Pressure | | | |
|---|---|---|---|---|---|---|---|---|
| | | | | Solvent | CO/O₂ | T | Temps | |
| | Catalyst | additive | Substrate | (ml) | (bar) | (° C.) | (h) | NCC |
| A3 | Ag(15%)/γ-Al₂O₃ | Et₃N | 25 mL | CH₃CN | 65/15 | 90 | 16 | 0 |
| | (4 mmol) | (1 mmol) | MeOH | 50 mL | (CO/O₂) | | | |
| | | TBAI | | | | | | |
| | | (1.5 mmol) | | | | | | |

### Reaction Step B—Hydrogenation

TABLE 10
Test conditions of hydrogenation with a Ag/γ-Al₂O₃
catalyst and the obtained results.

| | | | Solvent | Pressure | T | Time | |
|---|---|---|---|---|---|---|---|
| | Catalyst | Substrate | (ml) | H₂ (bar) | (° C.) | (h) | Yield (%)* |
| B3 | Ag(15 wt %)%)/γ-Al₂O₃ | 20 mmol | MeOH | 50 (H₂) | 200 | 16 | 47 (76% |
| | (4 mmol = 20 mol %) | DMO | 50 mL | | | | selectivity) |

*Hydrogenation yield is calculated using GC-MS, mesitylene is used as internal standard

### Example 17—Tests Carried Out with the Pd—Cu/Oxide Catalyst

Tables 11 and 12 below report respectively the test conditions of oxidative carbonylation (reaction step A) and of hydrogenation (reaction step B) with Pd—Cu catalysts on ZrO₂ and on γ-Al₂O₃ supports.

### Reaction Step A—Carbonylation

TABLE 11
Test conditions of oxidative carbonylation with Pd—Cu catalysts
on ZrO₂ and on γ-Al₂O₃ supports and the obtained results.

| | | | | | Pressure | | | |
|---|---|---|---|---|---|---|---|---|
| | | | | Solvent | CO/O₂ | T | Time | Obtained |
| | Catalyst | additives | Substrate | (ml) | (bar) | (° C.) | (h) | weight |
| A4-1 | Pd(2%)Cu(10%)- | Et₃N | 25 mL | CH₃CN | 65/15 | 90 | 16 | 2.2 g |
| | γ-Al₂O₃ | (1 mmol) | MeOH | 50 mL | (CO/O₂) | | | |
| | (0.24 mmol) | TBAI | | | | | | |
| | | (1.5 mmol) | | | | | | |
| A4-2 | Pd(2%)Cu(10%)- | Et₃N | 25 mL | CH₃CN | 65/15 | 90 | 16 | 1.4 g |
| | γ-Al₂O₃ | (1 mmol) | EtOH | 50 mL | (CO/O₂) | | | |
| | (0.24 mmol) | TBAI | | | | | | |
| | | (1.5 mmol) | | | | | | |
| A4-3 | Pd(2%)Cu(10%)- | Et₃N | 75 mL | | 65/15 | 90 | 16 | 3.71 g |
| | γ -Al₂O₃ | (1 mmol) | EtOH | | (CO/O₂) | | | |
| | (0.24 mmol) | TBAI | | | | | | |
| | | (1.5 mmol) | | | | | | |
| A4-5 | Pd(2%)Cu(10%)- | Et₃N | 25 mL | CH₃CN | 65/15 | 90 | 16 | 3.8 g |
| | ZrO₂ (0.24 mmol) | (1 mmol) | EtOH | 50 mL | (CO/O₂) | | | |
| | | TBAI | | | | | | |
| | | (1.5 mmol) | | | | | | |
| A4-6 | Pd(2%)Cu(10%)- | Et₃N | 75 mL | | 65/15 | 90 | 16 | 5.7 g |
| | ZrO₂ (0.24 mmol) | (1 mmol) | EtOH | | (CO/O₂) | | | |
| | | TBAI | | | | | | |
| | | (1.5 mmol) | | | | | | |

### Reaction Step B—Hydrogenation

TABLE 12
Test conditions of hydrogenation with Pd—Cu catalysts on
ZrO₂ and on γ-Al₂O₃ supports and the obtained results.

| | | | | Pressure H₂ | | Time | |
|---|---|---|---|---|---|---|---|
| | Catalyst | Substrate | Solvent (ml) | (bar) | T (° C.) | (h) | Yield (%) |
| B4-1 | Pd(2%)Cu(10%)- | 20 mmol | MeOH | 50 (H₂) | 200 | 16 | 57 (76% |
| | γ -Al₂O₃ (4 mmol) | DMO | 50 mL | | | | selectivity) |
| B4-2 | Pd(2%)Cu(10%)- | 20 mmol | EtOH | 50 (H₂) | 200 | 16 | 75% (80% |
| | γ-Al₂O₃ (4 mmol) | DEO | 50 mL | | | | selectivity) |
| B4-3 | Pd(2%)Cu(10%)- | 20 mmol | EtOH | 50 (H₂) | 200 | 16 | 70 (90% |
| | ZrO₂ (4 mmol) | DEO | 50 mL | | | | selectivity) |
| B4-4 | Pd(2%)Cu(10%)- | 20 mmol | EtOH | 50 (H₂) | 200 | 16 | 82 (90% |
| | ZrO₂ (5 mmol) | DEO | 50 mL | | | | selectivity) |
| B4-5 | Pd(2%)Cu(10%)- | 20 mmol | EtOH | 50 (H₂) | 200 | 16 | 90 (90% |
| | ZrO₂ (6 mmol) | DEO | 50 mL | | | | selectivity) |
| B4-6 | Pd(2%)Cu(10%)- | 20 mmol | EtOH | 40 (H₂) | 220 | 8 | 92 (94% |
| | ZrO₂ (4 mmol) | DEO | 50 mL | | | | selectivity) |

### Example 18—Tests Carried Out with Pd—Ag/γ-Al₂O₃ or SiO₂ Catalyst

Tables 13 and 14 below report respectively the test conditions of oxidative carbonylation (reaction step A) and of hydrogenation (reaction step B) with Pd—Ag/γ-Al₂O₃ or SiO₂ catalyst.

### Reaction Step A—Carbonylation

TABLE 13
Test conditions of oxidative carbonylation tests with Pd—Ag/γ-Al₂O₃
or SiO₂ catalyst SiO₂ supports and the obtained results.

| | | | | | Pressure | | | |
|---|---|---|---|---|---|---|---|---|
| | | | | Solvent | CO/O₂ | T | Time | Obtained |
| | Catalyst | Additive | Substrate | (ml) | (bar) | (° C.) | (h) | weight |
| A5-1 | Pd(2%)Ag(10%)- | Et₃N | 50 mL | CH₃CN | 65/15 | 90 | 16 | 3.5 g |
| | γ-Al₂O₃ | (1 mmol) | MeOH | 100 mL | (CO/O₂) | | | |
| | (0.24 mmol) | TBAI | | | | | | |
| | | (1.5 mmol) | | | | | | |
| A5-2 | Pd(2%)Ag(10%)- | Et₃N | 50 mL | CH₃CN | 65/15 | 90 | 16 | 3.1 g |
| | SiO₂ (0.24 mmol) | (1 mmol) | MeOH | 100 mL | (CO/O₂) | | | |
| | | TBAI | | | | | | |
| | | (1.5 mmol) | | | | | | |

### Reaction Step B—Hydrogenation

TABLE 14
Test conditions of hydrogenation with Pd—Ag catalysts on
γ-Al₂O₃ or SiO₂ supports and the obtained results.

| | | | | Pressure H₂ | | Time | |
|---|---|---|---|---|---|---|---|
| | Catalyst | Substrate | Solvent (ml) | (bar) | T (° C.) | (h) | Yield (%) |
| B5-1 | Pd(2%)Ag(10%)- | 20 mmol | MeOH | 50 (H₂) | 200 | 16 | 30 (37% |
| | γ-Al₂O₃ (4 mmol) | DMO | 50 mL | | | | selectivity) |
| B5-2 | Pd(2%)Ag(10%)- | 20 mmol | MeOH | 50 (H₂) | 200 | 16 | 7 (12% |
| | SiO₂ (4 mmol) | DMO | 50 mL | | | | selectivity) |

### Example 19—General Catalyst Recycling Procedure after the Hydrogenation of Dialkyl Oxalates

After one hydrogenation reaction of dialkyl oxalates, the catalyst (Cu or Ag) is separated from the liquid reaction medium by filtration. The catalyst was then washed with 3×25 mL ethanol. The material was then dried at 80° C. for 4 h before being used again in a hydrogenation reaction of dialkyl oxalate.

### Example 20—Catalyst Recycling Tests after the Hydrogenation of Dialkyl Oxalates

Tables 15 and 16 below report respectively the test conditions of hydrogenation (reaction step B) and the recycling results according to example 19.

TABLE 15
Test conditions of hydrogenation with a Pd—Cu/ZrO₂ catalyst and the obtained results.

| | | | | Pressure H₂ | | | |
|---|---|---|---|---|---|---|---|
| | Catalyst | Substrate | Solvent (ml) | (bar) | T (° C.) | Time (h) | Yield (%) |
| B6-1 | Pd(2%)Cu(10%)- | 20 mmol | MeOH | 50 (H₂) | 200 | 16 | 82% (90% |
| | ZrO₂ (5 mmol) | DMO | 50 mL | | | | selectivity) |

TABLE 16
Recycling results of hydrogenation
tests using a Pd—Cu/ZrO₂ catalyst.

| Cycles | Yield |
|---|---|
| 1 | 90% |
| | (90% selectivity) |
| 2 | 90% |
| | (90% selectivity) |
| 3 | 78% |
| | (77% selectivity) |

### Example 21: Optimisation of the Carbonylation Reaction with PdCu/ZrO₂

Table 17 below reports the results of oxidative carbonylation tests (reaction step A) with variations in the conditions (nature of the base, additive, O/CO₂ pressure, reaction time, quantity of catalyst and substrate) compared with the general procedure according to example 12 with a Pd(2%)Cu(10%)/ZrO₂ catalyst prepared according to example 5.

The carbonylation yield was calculated using GC-MS, with chlorobenzene used as an internal standard.

TABLE 17
Test conditions of the oxidative carbonylation with a
Pd(2%)Cu(10%)/ZrO₂ catalyst and the obtained results.

| | | Base | Additive | O₂/CO | T, | Time, | | |
|---|---|---|---|---|---|---|---|---|
| Reaction | Cat | (mmol) | (mmol) | (bar) | (° C.) | (h) | Scale | Results |
| A4-6 | Pd(2%)-Cu(10%)- | Et₃N (1) | TBAI | 15/65 | 90 | 16 | 75 mL | 5.7 g |
| | ZrO₂ (0.24 mmol) | | (1.5) | | | | EtOH | NCC = 162 |
| A6-1 | Pd(2%)-Cu(10%)- | Et₃N (2) | TBAI | 15/65 | 90 | 8 | 50 mL | 3.05 g |
| | ZrO₂ (0.24 mmol) | | (1.5) | | | | EtOH | NCC = 87 |
| A6-2 | Pd(2%)-Cu(10%)- | 2,6-lutidine | TBAI | 15/65 | 90 | 8 | 50 mL | 2.5 g |
| | ZrO₂ (0.24 mmol) | (2) | (1.5) | | | | EtOH | NCC = 71 |
| A6-3 | Pd(2%)-Cu(10%)- | Cs₂ CO₃ | TBAI | 15/65 | 90 | 8 | 50 mL | 1.23 g |
| | ZrO₂ (0.24 mmol) | (2) | (1.5) | | | | EtOH | NCC = 35 |
| A6-4 | Pd(2%)-Cu(10%)- | 1-Methyl- | TBAI | 15/65 | 90 | 8 | 50 ml | 1.3 g |
| | ZrO₂ (0.24 mmol) | imidazol | (1.5) | | | | EtOH | NCC = 37 |
| | | (2) | | | | | | |
| A6-5 | Pd(2%)-Cu(10%)- | Et₃N (2) | TBAI | 15/65 | 90 | 8 | 50 mL | 1.7 g |
| | ZrO₂ (0.24 mmol) | | (0.5) | | | | EtOH | NCC = 48 |
| A6-6 | Pd(2%)-Cu(10%)- | Et₃N (2) | CuCl₂ | 15/65 | 90 | 8 | 50 mL | 0.61 g |
| | ZrO₂ (0.24 mmol) | | (1.5) | | | | EtOH | NCC = 17 |
| A6-7 | Pd(2%)-Cu(10%)- | Et₃N (2) | FeCl₃ | 15/65 | 90 | 8 | 50 mL | 1.4 g |
| | ZrO₂ (0.24 mmol) | | (1.5) | | | | EtOH | NCC = 40 |
| A6-8 | Pd(2%)-Cu(10%)- | Et₃N (2) | NaI | 15/65 | 90 | 8 | 50 mL | 3.8 g |
| | ZrO₂ (0.24 mmol) | | (1.5) | | | | EtOH | NCC = 108 |
| A6-9 | Pd(2%)-Cu(10%)- | Et₃N (2) | KI | 15/65 | 90 | 8 | 50 mL | 3.23 g |
| | ZrO₂ (0.24 mmol) | | (1.5) | | | | EtOH | NCC = 92 |
| A6-10 | Pd(2%)-Cu(10%)- | Et₃N (2) | CaI₂ | 15/65 | 90 | 8 | 50 mL | 3.3 g |
| | ZrO₂ (0.24 mmol) | | (1.5) | | | | EtOH | NCC = 94 |
| A6-11 | Pd(2%)-Cu(10%)- | Et₃N (2) | NaI | 15/65 | 90 | 8 | 50 mL | 2.34 g |
| | ZrO₂ (0.12 mmol) | | (1.5) | | | | EtOH | NCC = 67 |
| A6-12 | Pd(2%)-Cu(10%)- | Et₃N (2) | NaI | 10/65 | 90 | 8 | 50 mL | 3.4 g |
| | ZrO₂ (0.24 mmol) | | (1.5) | | | | EtOH | NCC = 97 |
| A6-13 | Pd(2%)-Cu(10%)- | Et₃N (2) | NaI | 5/65 | 90 | 8 | 50 ml | 3.1 g |
| | ZrO₂ (0.24 mmol) | | (1.5) | | | | EtOH | NCC = 88 |

### Example 22: Volume Optimisation

Tests were carried out in a one-litre reactor.

### Test A-7-2

A heterogeneous catalyst based on palladium Pd(2%)Cu(10%)/ZrO₂ (0.54 mmol Pd), NaI (0.252 g, 1.68 mmol), tri-ethylamine (0.630 mL, 4.5 mmol), and ethanol (225 mL) were introduced into a 1 L Parr autoclave equipped with a magnetic stirrer. The reactor was sealed and the reaction mixture was purged three times with nitrogen (5 bar), and twice with oxygen (5 bar). The autoclave was then pressurised with 15 bar of oxygen and a further 65 bar of carbon monoxide (total pressure 80 bar). The reaction mixture was then stirred at 90° C. for 16 h. Once the reaction was complete, the autoclave was allowed to return to room temperature before being depressurised and purged three times with nitrogen (5 bar). The reaction mixture was then filtered and the recovered solution transferred to a 500 mL flask. The reaction solvent and the excess alcohol were separated by evaporation on a rotary evaporator. The oxalate was recovered after purification (vacuum distillation at 120° C./50-20 mbar for the diethyloxalate) and the isolated yields were calculated.

Tests A7-1 and A7-3 were carried out with variations in the concentrations of the catalyst, base, additive or substrate (ethanol) compared with the preparation conditions for test A7-2 described above.

Table 18 below reports the conditions and results of oxidative carbonylation tests (reaction step A) in a one-litre Parr autoclave, using a Pd(2%)Cu(10%)/ZrO₂ catalyst prepared according to example 5.

TABLE 18
Conditions and results of oxidative carbonylation tests in a one-litre Parr autoclave.

| | | Base | Additives | O₂/CO | T, | Time, | | |
|---|---|---|---|---|---|---|---|---|
| Reaction | Cat | (mmol) | (mmol) | (bar) | (° C.) | (h) | Scale | Results |
| A7-1 | Pd(2%)- | Et₃N | NaI | 15/65 | 90 | 16 | 225 mL | 13 g |
| | Cu(10%)-ZrO₂ | (9) | (6.75) | | | | EtOH | NCC = 82 |
| | (1.08 mmol) | | | | | | | After |
| | | | | | | | | distillation |
| A7-2 | Pd(2%)- | Et₃N | NaI | 15/65 | 90 | 16 | 225 mL | 13 g |
| | Cu(10%)-ZrO₂ | (4.5) | (1.68) | | | | EtOH | NCC = 165 |
| | (0.54 mmol) | | | | | | | After |
| | | | | | | | | distillation |
| A7-3 | Pd(2%)- | Et₃N | NaI | 15/65 | 90 | 16 | 150 mL | 10.5 g |
| | Cu(10%)-ZrO₂ | (4.5) | (3.37) | | | | EtOH | NCC = 133 |
| | (0.54 mmol) | | | | | | | After |
| | | | | | | | | distillation |

### Example 23: Influence of the Support and Preparation of PdCu/ZrO₂ Catalysts

In order to determine the influence of the ZrO₂ support and of the preparation of the catalysts, Cat B and Cat C catalysts were produced using another zirconium dioxide support, ZrO₂, consisting of a monoclinic crystalline phase, called hereinafter ZrO₂ (monoclinic phase) marketed by Alpha Aesar.

The ZrO₂ support (monoclinic phase) is identical to that used by Yuqing et al (Chinese Journal of Catalysis, 36, 2015, 1552-1559).

### Catalyst Cat A

The Pd(2%)Cu(10%)/ZrO₂ catalyst, hereafter referred to as Cat A, prepared according to example 5 was compared with Cat B and Cat C catalysts of the same nature but using a support with different characteristics or a different preparation.

### Catalyst Cat B: Influence of the Support

The Pd(2%)Cu(10%)/ZrO₂ (monoclinic phase), hereinafter referred to as Cat B, was prepared according to example 5 using the ZrO₂ support (monoclinic phase), supplied by Alfa Aesar (Thermo Fisher Scientific) with product number 43815, having a specific surface area greater than 85 m²/g.

### Catalyst Cat C: Influence of Preparation

The Pd(1%)Cu(3%)/ZrO₂ catalyst (monoclinic phase), referred to hereafter as Cat C, was prepared with the ZrO₂ support (monoclinic phase), supplied by Alfa Aesar, according to that described by the article Yuqing Jia et al. (Chinese Journal of Catalysis, 36, 2015, 1552-1559), namely in two successive steps:
an initial impregnation step with a palladium salt followed by calcination in air at 350° C.
a second impregnation step with a copper salt followed by calcination in air at 350° C.
   Unlike Yuqing Jia et al, a final reduction step was not carried out under hydrogen flow.

### Example 24

Tables 19 and 20 below show respectively the conditions and yield results for the oxidative carbonylation (reaction step A) and hydrogenation (reaction step B) tests using Cat A, Cat B and Cat C catalysts.

TABLE 19
Conditions of oxidative carbonylation tests with Cat
A, Cat B and Cat C catalysts and obtained results

| | | Base | Additives | O₂/CO | T | Time | | |
|---|---|---|---|---|---|---|---|---|
| Reaction | Cat | (mmol) | (mmol) | (bar) | (° C.) | (h) | Scale | Results |
| A4-6 | Cat A: Pd(2%)- | EtN₃ | TBAI | 15/65 | 90 | 16 | 75 mL | 5.7 g |
| | Cu(10%)-ZrO₂ | (1) | (1.5) | | | | EtOH | NCC = 162 |
| | (0.24 mmol) | | | | | | | |
| A8-1 | Cat B: Pd(2%)- | EtN₃ | TBAI | 15/65 | 90 | 16 | 75 mL | 4.9 g |
| | Cu(10%)-ZrO₂ | (1) | (1.5) | | | | EtOH | NCC = 140 |
| | (monoclinic phase) | | | | | | | |
| | (0.24 mmol) | | | | | | | |
| A8-2 | Cat C: Pd(1t %)- | EtN₃ | TBAI | 15/65 | 90 | 16 | 75 mL | 4.16 g |
| | Cu(3t %)-ZrO₂ | (1) | (1.5) | | | | EtOH | NCC = 119 |
| | (monoclinic phase) | | | | | | | |
| | (0.24 mmol) | | | | | | | |

TABLE 20
Conditions of hydrogenation tests with Cat A, Cat
B and Cat C catalysts and the obtained results

| | | | | | T | Time | |
|---|---|---|---|---|---|---|---|
| | Cat | Substrate | Solvent (ml) | Gas (bar) | (° C.) | (h) | Yield (%) |
| B4-6 | Cat A: Pd(2%)Cu(10%)- | 20 mmol | EtOH | 40 (H₂) | 220 | 8 | 92 (94% |
| | ZrO₂ (4 mmol) | DEO | 50 mL | | | | selectivity) |
| B8-1 | Cat B: Pd(2%)Cu(10%)- | 20 mmol | EtOH | 40 (H₂) | 220 | 8 | 61% (70% |
| | ZrO₂ (monoclinic | DEO | 50 mL | | | | selectivity) |
| | phase) (4 mmol) | | | | | | |
| B8-2 | Cat C: Pd(1%)Cu(3%)- | 20 mmol | EtOH | 40 (H₂) | 220 | 8 | 37% (35% |
| | ZrO₂ (monoclinic | WD | 50 mL | | | | selectivity) |
| | phase) (4 mmol) | | | | | | |

These results indicate a higher yield of the carbonylation of ethanol to diethyloxalate (step A) with a yield of 5.7 g and of the hydrogenation of oxalate to ethylene glycol (step B) with a yield of 92% and a selectivity of 94% for catalyst Cat A of the invention. 10

Cat B and Cat C catalysts, prepared with a ZrO₂ support from Alfa Aesar, are less efficient for carbonylation in terms of yield, but also for the hydrogenation step, with yields of 61% and 37% and selectivities of 70% and 35% respectively. In particular, Cat C catalyst prepared according to Yuqing Jia et al. is the least efficient in terms of carbonylation yield (step A) and in particular in terms of hydrogenation yield and selectivity (step B), which are 2.5 times lower than those of Cat A.

### Example 24: Analysis of PdCu/ZrO₂ Catalysts—Specific Surface Area

The specific surface area of Cat A, Cat B and Cat C catalysts was measured by BET. The results are shown in Table 21 below.

TABLE 21
Specific surface area of catalysts by BET

| | Specific surface |
|---|---|
| Catalyst | area BET |
| Cat A : | 5.64 m²/g |
| Pd (2%) Cu(10%)/ ZrO₂ | |
| Cat B | 62.85 m²/g |
| Pd (2%) Cu(10%)/ ZrO₂ (monoclinic phase) | |
| Cat C : | 81.92 m²/g |
| Pd (1%) Cu(3%)/ ZrO₂ (monoclinic phase) prepared | |
| according to Jia et al. | |

### Example 25: Structural Analysis of PdCu/ZrO₂ Catalysts

### a) Structure—Phase Analysis

X-ray powder diffractograms of Cat A, Cat B and Cat C catalysts were carried out using a Rigaku MINIFLEX II diffractometer, which emits X-rays via a tube and a copper source (wavelength Kα 1.54 Å).

FIGS. 3 and 4 represent the X-ray diffractograms obtained for Cat A, Cat B and Cat C catalysts respectively.

The results of the analysis of the various diffractograms obtained for Cat A, Cat B and Cat C catalysts are presented respectively in Tables 22, 23 and 24 below.

TABLE 22
Phase analysis of Cat A catalyst

| Crystalline phases identified | Crystalline phases | ICDD |
|---|---|---|
| by the database | identified | sheet |
| Cat A | Baddeleyite - ZrO₂ | 00-037-1484 |
| Pd(2%)Cu(10%)ZrO₂ | Copper monoxide - CuO | 01-089-2531 |
| (Sterm) | Palladium oxide - PdO | 00-043-1024 |
| | Palladium, syn - Pd | 01-087-0637 |

TABLE 23
Phase analysis of Cat B catalyst

| Crystalline phases identified | Crystalline phases | ICDD |
|---|---|---|
| by the database | identified | sheet |
| Cat B | Baddeleyite - ZrO₂ | 00-037-1484 |
| Pd(2%)Cu(10%)ZrO₂ (mono- | Copper monoxide - CuO | 01-089-2531 |
| clinic phase) | Palladium oxide - PdO | 00-043-1024 |
| | Palladium, syn - Pd | 01-087-0637 |

TABLE 24
Phase analysis of Cat C catalyst

| Crystalline phases identified | Crystalline phases | ICDD |
|---|---|---|
| by the database | identified | sheet |
| Cat C | Baddeleyite - ZrO₂ | 00-037-1484 |
| Pd(1%)Cu(3%)ZrO₂ (monoclinic | | |
| phase) | Palladium oxide - PdO | 00-043-1024 |

The diffractograms show the presence of crystallised phases.

The XRD analyses indicate that the same crystalline baddeleyite phase (ZrO₂) is present in all three catalysts with the presence of a palladium oxide phase. In the case of Cat A and Cat B catalysts, copper monoxide and palladium phases in metallic state are observed.

The diffraction peaks of Cat A and Cat B catalysts can be distinguished from the peaks of the Baddeleyite phase of the support. Cat A peaks are narrower in width than Cat B peaks.

### b) Structure—Crystallinity

### Crystallite Size

The crystallinity of materials is characterised by the size of the crystallites.

The crystallite size was qualitatively estimated in order to compare the different ZrO₂ supports of Cat A and Cat B catalysts. As a reminder, Cat B and Cat C catalysts were prepared using ZrO₂ support identical to that used in Yuqing Jia et al. (Chinese Journal of Catalysis, 36, 2015, 1552-1559) marketed by Alfa Aesar.

The size of the crystallites was evaluated using the following Scherrer formula:

$$t = \frac{k \cdot λ}{H \cdot cos θ}$$

t=crystallite size
k=correction factor=0.89
λ=wavelength of the source
H=width at half-height of peak (in radians)
θ=diffraction angle

The width at half-height was estimated using ImageJ processing software (developed by the National Institutes of Health).

The crystallite size calculations from Cat A and Cat B diffractograms are shown in Table 25 below:

TABLE 25
Crystallite sizes

| | | Crystallite |
|---|---|---|
| | | sizes |
| Reference | | (nm) |
| Cat A | Pd(2%)Cu(10%) / ZrO₂ of Sterm | 31.4 nm |
| Cat B | Pd(2%)Cu(10%) / ZrO₂ (monoclinic phase) from | 9.3 |
| | Alfa Aesar | |

The support used in Yuqing Jia et al. is a commercial ZrO₂ oxide powder from Alfa Aesar, with a pore volume of 0.27 cc/g and a specific surface area more than 85 m²/g (BET).

The low value of the sizes, in particular less than 10 nanometres, is an indicator of a low crystallinity structure. The smaller the crystallites, the wider the diffraction peaks. This effect becomes visible for crystallites less than 1 μm in diameter.

The results indicate that the catalyst prepared with Sterm's ZrO₂ support and the catalysts prepared with Alfa Aesar's ZrO₂ support have crystallite sizes of 31 nm and 9 nm respectively. Cat A and Cat B catalysts are thus distinguished by the microstructure of ZrO₂ support.

In addition to the characteristic related to the specific surface area, these results show that Cat A and Cat B catalyst supports are different in terms of their microstructure.

Thus, as shown in Examples 17, 21 and 24, catalysts prepared with a ZrO₂ support having a crystallite size of about 30 nm used for steps A and B of ethylene glycol preparation are more efficient than PdCu/ZrO₂ catalysts prepared with a more polycrystalline and lower crystallinity ZrO₂ support having a crystallite size of about 9 nm.

### Example 26: Morphology and Composition Analysis

The SEM images in FIGS. 2, 5, 6, 7 and 8 were taken using a Zeiss MEB-FEG scanning microscope with controlled pressure, making it possible to observe materials with little or no conductivity without any specific preparation.

The sample was stabilised on carbon adhesive paper to enable SEM observation. It should therefore be noted that the content of the element carbon can be associated with the use of the latter.

Quantification was carried out by EDX spectrum analysis on sample areas of the catalysts. The results are expressed as a percentage by mass.

The results of the SEM observations and EDX analysis are summarised below.

### Cat A: Pd(2%)Cu(10%)/ZrO₂ (Sterm)

SEM images are shown in FIGS. 2 and 5. Entangled elongated particles with heterogeneous thicknesses of less than a micrometre can be seen. These particles form clusters of a few micrometres to a hundred micrometres.

The EDX analysis of one zone of the sample is shown in Table 26. The particles are composed mainly of zirconium (Zr), oxygen (O) and copper (Cu) and to a lesser extent of palladium (Pd), hafnium (Hf) and carbon.

TABLE 26
Chemical composition of Cat A catalyst

| | Quantification using the |
|---|---|
| | EDX spectrum |
| Element | (in % by mass) |
| C | 1.32 |
| O | 24.89 |
| Zr | 62.91 |
| Pd | 1.42 |
| Cu | 7.91 |
| Hf | 1.56 |
| Total | 100.00 |

### Catalyst: Pd(2%)/ZrO₂ (Sterm)

SEM observations of Pd(2%)/ZrO₂ catalyst prepared with Sterm's ZrO₂ support reveal a morphology similar to that of Cat A catalyst, namely entangled elongated particles of heterogeneous thickness less than one micrometre forming clusters.

### Cat B: Pd(2%)Cu(10%)/ZrO₂ (Monoclinic Phase) Alfa Aesar

FIG. 6 shows SEM images of the Cat B catalyst. The SEM observations reveal the presence of a population of macroscopic particles with a polyhedral morphology of a few hundred micrometres, with smaller particles with a spherical morphology of a few tens of nanometres on the surface.

The EDX analysis of two zones of the sample is shown in Table 27.

These particles are composed mainly of copper (Cu), zirconium (Zr), palladium (Pd) and oxygen (O), with a minority of carbon (C) and traces of hafnium (Hf) and rhenium (Re).

TABLE 27
Chemical composition of Cat B catalyst

| | Zone 1 - Quantification by | Zone 2- Quantification |
|---|---|---|
| | EDX spectrum (in % by | using the EDX spectrum |
| Element | mass) | (in % by mass) |
| C | 2.82 | 1.42 |
| O | 16.66 | 17.01 |
| Zr | 26.26 | 37.31 |
| Pd | 13.97 | 7.13 |
| Cu | 38.92 | 34.06 |
| Hf | 0.68 | 2.25 |
| Re | 0.68 | 0.83 |
| Total | 100.00 | 100.00 |

### Cat C: Pd(1%)Cu(3%)/ZrO₂ (Monoclinic Phase) Alfa Aesar Prepared According to Jia et al.

SEM observations reveal the presence of micrometric particles with a polyhedral morphology and nanometric particles.

The EDX analysis of one zone of the sample is shown in Table 28.

These particles are composed mainly of zirconium (Zr), oxygen (O) and copper (Cu), and to a lesser extent carbon (C), hafnium (Hf) and rhenium (Re), with traces of palladium (Pd).

TABLE 28
Chemical composition of Cat C catalyst

| | Quantification using the |
|---|---|
| | EDX spectrum |
| Element | (in % by mass) |
| C | 2.14 |
| O | 27.19 |
| Zr | 59.07 |
| Pd | 0.84 |
| Cu | 8.11 |
| Hf | 1.38 |
| Re | 1.28 |
| Total | 100.00 |

Catalyst Pd(2%)Cu(10%)/γ-Al₂O₃

FIG. 7 shows the SEM images of the Pd(2%)Cu(10%)/γ-Al₂O₃. SEM observations reveal the presence of a population of macroscopic particles with a generally spherical morphology, agglomerated to a size of a few tens of micrometres, with a porous, honeycomb-like morphology on the surface.

Catalyst Pd(2%)Ag(15%)/γ-Al₂O₃

FIG. 8 shows the SEM images of the Pd(2%)Ag(15%)/γ-Al₂O₃. SEM observations reveal the presence of a population of macroscopic particles with a generally spherical morphology, agglomerated to a size of a few tens of micrometres, which have a porous morphology with a honeycomb appearance on the surface. The particles have the same appearance and morphology as the Pd(2%)Cu(10%)/v-Al₂O₃.

### Example 27: Surface Study of XPS Catalysts

Analyses are carried out using a PHI QUANTES photoemission spectrometer. This instrument is equipped with a monochromate X-ray source (aluminium K_{α} line) as well as a chromium X-ray source for Hard XPS, a charge neutralisation system for electrically insulating samples and a hemispherical electron analyser.

XPS analyses were carried out on:
Cat A: Pd(2%)Cu(10%)/ZrO₂ (Sterm) (see spectra in FIG. 1)
Pd(2%)/ZrO₂ catalyst (Sterm) (see spectrum in FIG. 9)
Pd(2%)Cu(10%)/γ-Al₂O₃ (see spectrum in FIG. 10)
Pd(2%)Ag(15%)/v-Al₂O₃ (see spectrum in FIG. 11)

### Cat A

### Cu2p Spectrum

Analysis of the XPS spectra over several zones reveals a variation of the satellite typical for CuO (around 940-945 eV), indicating the presence of a mixture of Cu+ and Cu2+.

The Cu+ concentration is higher because Auger signal is dominated by this component (916.8 eV). The presence of metallic Cu cannot be detected either on the Cu2p spectrum or on the Auger spectrum. If it is present, it is masked by the signals corresponding to Cu+.

### Pd3d Spectrum

Pd3d is interfered by Zr3p signal. A reference ZrO₂ powder was measured in order to extract the Zr3p signal, i.e. Zr3p3 at 332.9 eV and Zr3p1 at 346.61 eV.

The presence of Pd2+ is confirmed by the corresponding Pd3d5/2 signal (located at 337.5 eV) which is visible.

### Pd(2%)/ZrO₂ Catalyst (Sterm) (See FIG. 9)

### Pd3d Spectrum

The Zr3d spectra overlap, at least in energy. The width is slightly greater, which may be due to differential charging. This correspondence of binding energies should be found in the case of Pd3d.

Pd3d is interfered by Zr3p signal. The presence of Pd2+ is certain, as the corresponding Pd3d5/2 signal (located at 337 eV) is visible.

Pd(2%)Cu(10%)/γ-Al₂O₃ (See Spectrum in FIG. 10 and Table 29 Below)

### Cu2p Spectrum

Cu2p spectra mainly show the presence of Cu2+ in the form of oxide and hydroxide (based on the appearance of the satellite).

### Pd3d Spectrum

Pd3d spectra are identical for the two zones measured.

Pd3d5/2 component is located at 337.5 eV.

TABLE 29
Binding energies of XPS spectrum
of the Pd(2%) Cu(10%)/ γ-Al₂O₃

| | Peak |
|---|---|
| Name | (Binding energy) |
| Cu2p3 | 935.08 |
| Pd3d | 337.49 |
| Al2s | 119.64 |

Pd(2%)Ag(15%)/γ-Al₂O₃ (See Spectrum in FIG. 11 and Table 30 Below)

### Ag3d Spectrum

Ag3d spectrum was difficult to interpret and required comparison with reference spectra (metallic Ag spectrum). An analysis of the Auger signals shows that it is a mixture of Ag oxides (I and II).

### Pd3d Spectrum

For Pd3d spectrum, the energy position is slightly lower at 337.2 eV, with an additional component at lower binding energy; Pd3d peaks for the sample are wider. There is therefore a difference of 0.3 eV in the position of Pd5/2 peak between the two samples.

TABLE 30
Binding energies of the XPS spectrum of the catalyst

| | Peak |
|---|---|
| Name | (Binding energy) |
| Ag3d5 | 368.67 |
| Pd3d | 337.21 |
| Al2p | 74.77 |

1.-15. (canceled)

16. A method of preparation of ethylene glycol comprising:
a first reaction step A of oxidative carbonylation of an alcohol, in the presence of a supported bimetallic catalyst of formula Pd-M/Support, comprising palladium and a metal M on a support, in which M represents Cu or Ag, to obtain an oxalate compound as reaction intermediate, and
a second reaction step B of hydrogenation of said oxalate compound, optionally purified, produced in reaction step A, to ethylene glycol, in the presence of a catalyst of formula Pd-M/Support.

17. The method of preparation of ethylene glycol according to claim 16, wherein said method comprising two reaction steps catalysed by the same bimetallic catalyst,
in which the first catalysed reaction step A is an oxidative carbonylation, from an alcohol, carbon monoxide and an oxidant, in particular molecular oxygen, optionally in the presence of a promoter, to form an oxalate compound as reaction intermediate, and
in which the second catalysed reaction step B is a hydrogenation reaction of the said oxalate compound with hydrogen to obtain ethylene glycol.

18. The method of preparation of ethylene glycol according to claim 16, wherein:
reaction step A comprises
bringing into contact:
an alcohol, in particular selected from methanol and ethanol,
carbon monoxide,
an oxidant, in particular oxygen O₂,
a catalyst of formula Pd-M/Support in which M represents Cu or Ag,
optionally a promoter, in particular an iodine compound, notably selected from tetramethylammonium iodide, potassium iodide or sodium iodide, preferably tetramethylammonium iodide,
optionally a base, in particular triethylamine,
optionally a solvent, in particular selected from acetonitrile, tetrahydrofuran, dioxane and toluene, preferably acetonitrile;
to obtain a reaction medium 1 which may be pressurised from 0.1 to 15 MPa, optionally heating said reaction medium 1 to a temperature from 25 to 200° C.,
preferably about 90° C., in particular during from 2 to 24 hours, preferably 16 hours, to obtain the oxalate compound;
reaction step B comprises
bringing into contact:
said oxalate compound,
dihydrogen,
said catalyst of formula Pd-M/Support in which M represents Cu or Ag,
optionally a solvent, in particular ethanol, methanol and dioxane, preferably ethanol or methanol,
to obtain a reaction medium 2 which may be pressurised from 0.1 to 15 MPa, in particular 5 MPa,
optionally heating said reaction medium 2 to a temperature from 100 to 250° C., in particular 200 or 220° C., preferably during from 5 to 24 hours, more preferably for 8 or 16 hours,
to obtain ethylene glycol.

19. The method of preparation of ethylene glycol according to claim 16, wherein:
reaction step A comprises
bringing into contact:
an alcohol, in particular selected from methanol and ethanol, carbon monoxide,
an oxidant, in particular oxygen O₂,
a catalyst of formula Pd-M/Support in which M represents Cu or Ag,
a promoter, in particular an iodine compound, notably selected from tetramethylammonium iodide, potassium iodide or sodium iodide, preferably tetramethylammonium iodide,
a base, in particular triethylamine,
a solvent, in particular selected from acetonitrile, tetrahydrofuran, dioxane and toluene, preferably acetonitrile;
to obtain a reaction medium 1 which may be pressurised from 0.1 to 15 MPa,
heating said reaction medium 1 to a temperature from 25 to 200° C., during from 2 to 24 hours,
to obtain the oxalate compound;
reaction step B comprises
bringing into contact:
said oxalate compound,
dihydrogen,
said catalyst of formula Pd-M/Support in which M represents Cu or Ag,
a solvent, in particular ethanol, methanol and dioxane, preferably ethanol or methanol,
to obtain a reaction medium 2 which may be pressurised from 0.1 to 15 MPa, in particular 5 MPa,
heating said reaction medium 2 to a temperature from 100 to 250° C., preferably during from 5 to 24 hours,
to obtain ethylene glycol.

20. The method of preparation of ethylene glycol according to claim 16, wherein:
reaction step A comprises
bringing into contact:
an alcohol, in particular selected from methanol and ethanol,
carbon monoxide,
an oxidant, in particular oxygen O₂,
a catalyst of formula Pd-M/Support in which M represents Cu or Ag,
a promoter, in particular an iodine compound, notably selected from tetramethylammonium iodide, potassium iodide or sodium iodide, preferably tetramethylammonium iodide,
a base, in particular triethylamine,
a solvent, in particular selected from acetonitrile, tetrahydrofuran, dioxane and toluene, preferably acetonitrile;
to obtain a reaction medium 1 pressurised from 0.1 to 15 MPa,
heating said reaction medium 1 to a temperature from 25 to 200° C., during from 2 to 24 hours,
to obtain the oxalate compound;
reaction step B comprises
bringing into contact:
said oxalate compound,
dihydrogen,
said catalyst of formula Pd-M/Support in which M represents Cu or Ag,
a solvent, in particular ethanol, methanol and dioxane, preferably ethanol or methanol,
to obtain a reaction medium 2 pressurised from 0.1 to 15 MPa, in particular 5 MPa,
heating said reaction medium 2 to a temperature from 100 to 250° C., during from 5 to 24 hours,
to obtain ethylene glycol.

21. The method of preparation of ethylene glycol according to claim 16, wherein:
reaction step A comprises
bringing into contact:
an alcohol, in particular selected from methanol and ethanol, carbon monoxide,
oxygen O₂,
a catalyst of formula Pd-M/Support in which M represents Cu or Ag,
a promoter selected from tetramethylammonium iodide, potassium iodide or sodium iodide, preferably tetramethylammonium iodide,
to obtain a reaction medium 1 pressurised from 0.1 to 15 MPa,
heating said reaction medium 1 to a temperature from 25 to 200° C., during from 2 to 24 hours,
to obtain the oxalate compound;
reaction step B comprises
bringing into contact:
said oxalate compound,
dihydrogen,
said catalyst of formula Pd-M/Support in which M represents Cu or Ag,
a solvent, in particular ethanol, methanol and dioxane, preferably ethanol or methanol,
to obtain a reaction medium 2 pressurised from 0.1 to 15 MPa, in particular 5 MPa,
heating said reaction medium 2 to a temperature from 100 to 250° C., during from 5 to 24 hours,
to obtain ethylene glycol.

22. The method of preparation of ethylene glycol according to claim 16, wherein:
reaction step A comprises
bringing into contact:
an alcohol, in particular selected from methanol and ethanol,
carbon monoxide,
oxygen O₂,
a catalyst of formula Pd-M/Support in which M represents Cu or Ag,
a promoter,
a base, in particular triethylamine,
a solvent selected from acetonitrile, tetrahydrofuran, dioxane and toluene, preferably acetonitrile;
to obtain a reaction medium 1 pressurised from 0.1 to 15 MPa,
heating said reaction medium 1 to a temperature from 25 to 200° C., during from 2 to 24 hours,
to obtain the oxalate compound;
reaction step B comprises
bringing into contact:
said oxalate compound,
dihydrogen,
said catalyst of formula Pd-M/Support in which M represents Cu or Ag,
a solvent selected from ethanol, methanol and dioxane,
to obtain a reaction medium 2 pressurised from 0.1 to 15 MPa, in particular 5 MPa,
heating said reaction medium 2 to a temperature from 100 to 250° C., during from 5 to 24 hours,
to obtain ethylene glycol.

23. The method of preparation of ethylene glycol according to claim 16, wherein:
reaction step A comprises
bringing into contact:
an alcohol, in particular selected from methanol and ethanol,
carbon monoxide,
oxygen O₂,
a catalyst of formula Pd-M/Support in which M represents Cu or Ag, a promoter,
a base, in particular triethylamine,
to obtain a reaction medium 1 pressurised from 0.1 to 15 MPa,
heating said reaction medium 1 to a temperature from 25 to 200° C., during from 2 to 24 hours,
to obtain the oxalate compound;
reaction step B comprises
bringing into contact:
said oxalate compound,
dihydrogen,
said catalyst of formula Pd-M/Support in which M represents Cu or Ag,
a solvent selected from ethanol, methanol and dioxane,
to obtain a reaction medium 2 pressurised from 0.1 to 15 MPa, in particular 5 MPa,
heating said reaction medium 2 to a temperature from 100 to 250° C., during from 5 to 24 hours,
to obtain ethylene glycol.

24. The method of preparation of ethylene glycol according to claim 16, wherein:
reaction step A comprises
bringing into contact:
an alcohol, in particular selected from methanol and ethanol,
carbon monoxide,
oxygen O₂,
a catalyst of formula Pd-M/Support in which M represents Cu or Ag,
a promoter,
a base selected from triethylamine (Et₃N), 2,6-lutidine, caesium carbonate (Cs₂CO₃), or 1-methylimidazole, in particular triethylamine,
a solvent selected from acetonitrile, tetrahydrofuran, dioxane and toluene, preferably acetonitrile;
to obtain a reaction medium 1 pressurised from 0.1 to 15 MPa,
heating said reaction medium 1 to a temperature from 25 to 200° C., during from 2 to 24 hours,
to obtain the oxalate compound;
reaction step B comprises
bringing into contact:
said oxalate compound,
dihydrogen,
said catalyst of formula Pd-M/Support in which M represents Cu or Ag,
a solvent selected from ethanol, methanol and dioxane,
to obtain a reaction medium 2 pressurised from 0.1 to 15 MPa, in particular 5 MPa,
heating said reaction medium 2 to a temperature from 100 to 250° C., during from 5 to 24 hours,
to obtain ethylene glycol.

25. The method of preparation of ethylene glycol according to claim 16,
wherein reaction step A comprises bringing into contact an alcohol of Formula 1 to prepare an oxalate compound of Formula 2: 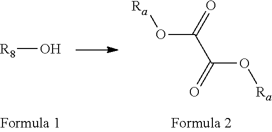
in which Rₐ represents:
a C₁ to C₂₀ linear or branched alkyl group,
a C₃ to C₁₀ cycloalkyl group,
a C₅ to C₂₀ alkyl-aryl or alkyl-heteroaryl group.

26. The method of preparation of ethylene glycol according to claim 16, wherein the alcohol of reaction step A is selected from methanol, ethanol and isopropanol.

27. The method of preparation of ethylene glycol according to claim 16, in which the catalyst support is an oxide selected from zirconium dioxide ZrO₂, alumina Al₂O₃, silica SiO₂, cerium dioxide CeO₂, titanium dioxide TiO₂, magnesium oxide MgO, indium oxide In₂O₃, or a mixture of these oxides, preferably zirconium dioxide ZrO₂.

28. The method of preparation of ethylene glycol according to claim 16, in which the catalyst has a palladium content from 0.1 to 10%, in particular 2%, and a content of metal M from 0.1 to 40%, in particular 10% or 15%, by weight relative to the total weight of the catalyst.

29. The method of preparation of ethylene glycol according to claim 16, wherein said supported bimetallic catalyst is a Pd—Cu/ZrO₂ catalyst comprising palladium and copper on a zirconium dioxide support.

30. A bimetallic catalyst of palladium and copper on a zirconium dioxide support of formula Pd—Cu/ZrO₂, comprising:
a palladium content from 0.1 to 10%, in particular 2%, and a copper content from 0.1 to 40%, in particular 10%, by weight relative to the total weight of the catalyst and, a surface area, measured by BET, from 1 to 50 m²/g, in particular from 1 to 10 m²/g, preferably from 5 to 7 m²/g.

31. The bimetallic catalyst according to claim 30, further comprising:
a crystalline phase of zirconium dioxide, analysed by X-ray diffraction, crystallised in monoclinic baddeleyite comprising a crystallite size from 20 to 100 nm, preferably from 20 to 50 nm, and optionally comprising hafnium atoms as an impurity.

32. The bimetallic catalyst according to claim 30, said catalyst being in the form of two populations of particles:
a first population of particles with a polyhedral morphology and
a second population of particles smaller in size than the first population and having a rounded and entangled morphology, with sizes from 10 nm to 1 micrometre, said particles forming clusters from 1 to 100 micrometres.

33. The bimetallic catalyst according to claim 30, further comprising:
copper atoms in oxidation state (I) and (II), and
palladium atoms in oxidation state (II),
in particular the molar quantity of copper atoms in an oxidation state (I) is greater than that of copper atoms in an oxidation state (II).

34. A method of preparation of a Pd—Cu/ZrO₂ catalyst, comprising:
a step C of impregnating a palladium salt and a copper salt, in particular palladium nitrate and copper nitrate, dissolved in an aqueous solution, in particular in a volume of water from 5 to 10 mL, on a zirconium dioxide support, in particular in powder form, with a ratio of solution mass/support mass from 0.6 and 1.0;
to obtain Pd—Cu/ZrO₂ catalyst in the form of a homogeneous material,
in particular said step C comprises:
the use of a palladium salt concentration calculated to obtain a palladium content from 0.1 to 10% by weight relative to the total weight of the catalyst,
the use of a copper salt concentration calculated to obtain a copper content from 0.1 to 40% by weight relative to the total weight of the catalyst,
a step D of drying said homogeneous material, in particular at a temperature from 60 to 100° C., in particular 80° C., preferably for a period from 10 to 24 hours, in particular 16 hours, to obtain the Pd—Cu/ZrO₂ catalyst in the form of an anhydrous homogeneous material,
an activation step E, in particular comprising calcination of said anhydrous homogeneous material at a temperature from 200 to 1000° C., in particular 600° C., preferably for a period from 1 to 15 hours, in particular 2 hours, in order to obtain said catalyst.

35. A bimetallic catalyst of palladium and copper on a zirconium dioxide support of formula Pd—Cu/ZrO₂, obtainable by a method according to claim 34.
